(19) United States
(12) Patent Application Publication
(10) Pub. No.: US 2016/0287152 A1
(43) Pub. Date: Oct. 6, 2016

(54) Functionalized Nanoparticles, Methods and In Vivo Diagnostic System

(71) Applicant: Verily Life Sciences LLC, Mountain View, CA (US)

(72) Inventors: Jerrod Joseph SCHWARTZ, Mountain View, CA (US); Krishnan Kanna PALANIAPPAN, San Francisco, CA (US); Alberto Clemente VITARI, San Francisco, CA (US)

(73) Assignee: Verily Life Sciences LLC

(21) Appl. No.: 15/083,830

(22) Filed: Mar. 29, 2016

Related U.S. Application Data
(60) Provisional application No. 62/140,302, filed on Mar. 30, 2015.

(51) Int. Cl. A61B 5/145; A61K 49/22; A61B 5/00; A61K 49/00

(52) U.S. Cl. CPC A61B 5/14546; A61K 49/0093; A61K 49/0054; A61K 49/222; A61K 49/225; A61B 5/681

(57) ABSTRACT

A nanoparticle conjugate includes a nanoparticle, first oligonucleotides of one or more types bound to the nanoparticle, each type of first oligonucleotide having a sequence, and targeting conjugates of one or more types, each type of targeting conjugate comprising a targeting entity and a second oligonucleotide bound to the targeting entity and having a sequence that is complementary to a sequence of a predetermined type of the first oligonucleotides, wherein the second oligonucleotide are covalently bound to a surface of the nanoparticle, a functional group on the surface of the nanoparticle, or one of the first oligonucleotide. Also provided are methods for making such nanoparticle conjugates and methods and devices for using such nanoparticle conjugates.

### CROSS-REFERENCE

This application claims the benefit of priority from U.S. Provisional Application No. 62/140,302, filed Mar. 30, 2015, which is incorporated by reference in its entirety.

### BACKGROUND

Unless otherwise indicated herein, the materials described in this section are not prior art to the claims in this application and are not admitted to be prior art by inclusion in this section.

A number of diagnostic methods have been developed to evaluate physiological conditions of a person by detecting and/or measuring one or more analytes in a person's blood or other bodily fluids or tissue. One or more target analytes could be any analytes that, when present in or absent from the blood, or present at a particular concentration or range of concentrations, may be indicative of a medical condition or health state of the person. These target analytes could include enzymes, reagents, hormones, proteins, cells, ions, e.g., sodium, potassium, calcium, or chloride, or molecules such creatine, urea, and carbohydrates, e.g., glucose. While many of the diagnostic methods that employ labeled agents are useful, they can be improved.

Much effort has been devoted into developing nanoparticles as vehicles for diagnosis, imaging as well as delivery of agents for treatment of disease. Nanoparticles have been conjugated to drugs, imaging agents or other substances that can be delivered to specific sites either by active targeting or by size-dependent passive targeting. However, one challenge associated with the use of nanoparticles particularly for simultaneous detection of multiple target analytes is developing chemical reactions between the nanoparticle and targeting entity, e.g., antibody, aptamer, peptide, small molecule, etc, that is both bioorthogonal and amenable to detection of multiple target analytes. Enhancing the robustness of nanoparticle conjugate probes for in vivo application is also another challenge. Accordingly, there is a need for improved, robust nanoparticle conjugates probes having a predetermined tailored set of properties that are particularly useful in diagnostic, imaging and/or therapeutic methods to determine or monitor a medical condition or a person's state of health.

### SUMMARY

One aspect of the present disclosure provides a composition. The composition includes: a nanoparticle; first oligonucleotides of one or more types that are bound to the nanoparticle, each type of first oligonucleotides having a sequence; and targeting conjugates of one or more types, each type of targeting conjugate comprising a targeting entity and a second oligonucleotide bound to the targeting entity and having a sequence that is complementary to the sequence of a predetermined type of the first oligonucleotides, wherein the second oligonucleotide is covalently bound to a surface of a nanoparticle, a functional group on the surface of the nanoparticle, or one of the first oligonucleotides. In some embodiments, the composition involves two or more types of targeting conjugates and two or more types of first oligonucleotide. In other embodiments, the composition involves a single type of first oligonucleotides and two or more types of targeting conjugates. In some embodiments, the targeting entity comprises a member of a specific binding pair.

In another aspect, the present disclosure provides a library of nanoparticle conjugates. The library includes nanoparticle conjugates of one or more types, each type of nanoparticle conjugate includes: a nanoparticle; first oligonucleotides of one or more types that are bound to the nanoparticle, each type of first oligonucleotides having a sequence; and targeting conjugates of one or more types, each type of targeting conjugate comprising a targeting entity and a second oligonucleotide bound to the targeting entity and having a sequence that is complementary to a sequence of a predetermined type of the first oligonucleotides, wherein the second oligonucleotide is covalently bound to a surface of the nanoparticle, a functional group on the surface of the nanoparticle, or one of the first oligonucleotides.

In another aspect, the present disclosure provides a method. The method includes: (a) providing a first nanoparticle conjugate of comprising a nanoparticle and first oligonucleotides of one or more types bound to the nanoparticles, each type of first oligonucleotides having a sequence; (b) contacting the first nanoparticle conjugate with targeting conjugates of one or more types, each type of targeting conjugate comprising a targeting entity and a second oligonucleotide bound to the targeting entity, wherein the contacting occurs under suitable hybridization conditions to form a second nanoparticle conjugate, wherein the second oligonucleotide has a sequence that is complementary to a sequence of the first oligonucleotides and wherein the second oligonucleotide include a reactive group that binds to a surface of the nanoparticle, a functional group on the nanoparticle, or one of the first oligonucleotides; (c) washing the second nanoparticle conjugate; and (d) covalently reacting the reactive group of second oligonucleotide of the second nanoparticle conjugate to the surface of the nanoparticles, the functional group on the nanoparticle, or one of the first oligonucleotides under suitable reaction conditions to form a third nanoparticle conjugate. In some embodiments, the second oligonucleotides are covalently bound to the surface or a functional group on the surface of the third nanoparticle conjugate.

In another aspect, the present disclosure provides another method. The method includes: (a) providing a first nanoparticle conjugate comprising a nanoparticle and a first oligonucleotide bound to the nanoparticles; (b) contacting the first nanoparticle conjugate with second oligonucleotides of one or more types, each type of second oligonucleotides comprising a targeting sequence and a sequence that is complementary to the sequence of the first oligonucleotide to produce a second nanoparticle conjugate, wherein said contacting occurs under suitable hybridization conditions; (c) extending the first oligonucleotide under suitable polymerase chain reaction conditions to produce an extended first oligonucleotide comprising a complementary sequence that is complementary to targeting sequence of the second oligonucleotide; (d) removing the second oligonucleotide from the second nanoparticle under suitable dehybridization conditions to produce a third nanoparticle conjugate; (e) washing the third nanoparticle conjugate; (f) contacting the third nanoparticle conjugate with a targeting conjugate of one or more types, each type of targeting conjugate comprising a targeting entity and a third oligonucleotide that is bound to the targeting entity, wherein the third oligonucleotide has a predetermined sequence that is complementary to the complementary targeting sequence of the extended first oligonucleotide, wherein the third oligonucleotide has a reactive group that is capable of covalently binding to a surface of the nanoparticle, a functional group on the nanoparticle, or the first oligonucleotide and wherein said contacting occurs under hybridization conditions to form a fourth nanoparticle conjugate. In some embodiments, the method further includes: (f1) covalently binding the third oligonucleotide to the surface of the nanoparticle, to a functional group on the nanoparticle, or to the first oligonucleotide to form a nanoparticle conjugate probe. In other embodiments, the method further includes: (g) extending the third oligonucleotide under suitable polymerase chain reaction conditions to produce a modified third oligonucleotide comprising a sequence that is complementary to the sequence of the first oligonucleotide; and (h) covalently reacting the reactive group of the modified third oligonucleotide with the surface of the nanoparticle or a functional group on the nanoparticle to form a nanoparticle conjugate probe. In some embodiments, the method involves a single type of second oligonucleotide and a single type of targeting conjugates. In other embodiments, the method involves two or more types of second oligonucleotides and two or more types of targeting conjugates.

In another aspect, the present disclosure provides a further method. The method includes: (a) providing a first nanoparticle conjugate comprising a nanoparticle and one type of oligonucleotides bound to the nanoparticles; (b) contacting the first nanoparticle conjugate with a second oligonucleotide of two or more types, each type of second oligonucleotides comprising a targeting sequence and a sequence that is complementary to a sequence of the first oligonucleotide to produce a second nanoparticle conjugate, wherein said contacting occurs under suitable hybridization conditions; (c) washing the second nanoparticle conjugate; (d) contacting the second nanoparticle conjugate with targeting conjugates of one or more types, each type of targeting conjugate comprising a targeting entity and a third oligonucleotide that is bound to the targeting entity, the third oligonucleotide having a predetermined sequence that is complementary to the targeting sequence of the second oligonucleotide, wherein said contacting occurs under suitable hybridization conditions to form a third nanoparticle conjugate; and (e) ligating the third oligonucleotide to the first oligonucleotide under suitable ligation conditions to form a fourth nanoparticle conjugate. In some embodiments, the method further comprises removing or melting off the second oligonucleotide of the fourth nanoparticle conjugate under suitable dehybridization conditions to form a nanoparticle conjugate probe. In other embodiments, the method further includes: covalently binding the second oligonucleotide of the fourth nanoparticle conjugate to form a nanoparticle conjugate probe. In some embodiments, the method involves a single type of second oligonucleotides and a single type of targeting conjugates. In some embodiments, the method involves two or more types of second oligonucleotide and two or more types of targeting conjugates.

In another aspect, the present disclosure provides a system. The system includes a wearable device comprising: a mount configured to mount the wearable device on an external surface of a living body; a detector configured to detect an analyte response signal transmitted from tissue through the external surface, wherein the tissue contains a nanoparticle conjugate comprising a nanoparticle; first oligonucleotides of one or more types that are bound to the nanoparticle, each type of first oligonucleotides having a sequence; and targeting conjugates of one or more types, each type of targeting conjugate comprising a targeting entity and a second oligonucleotide bound to the targeting entity and having a sequence that is complementary to the sequence of a predetermined type of the first oligonucleotides, wherein the second oligonucleotide are covalently bound to a surface of the nanoparticle, a functional group on the surface of the nanoparticle, or one of the first oligonucleotides; and a processor configured to determine a presence or absence of the one or more target analytes based on the analyte response signal. In one embodiment, the system further comprising a modulation source configured to modulate the analyte response signal differently than a background signal. In another embodiment, the processor is configured to differentiate the analyte response signal from the background signal based, at least in part, on the modulation by the modulation source. In some embodiments, the system further comprises an interrogating signal source configured to apply an interrogating signal to the tissue, wherein the analyte response signal is transmitted in response to the interrogating signal. In other embodiments, the modulation source is configured to modulate the analyte response signal by modulating the interrogating signal. In other embodiments, the tissue comprises subsurface vasculature, and wherein the particles are in blood circulating in the subsurface vasculature.

In a further aspect, the present disclosure provides a method. The method includes: (i) introducing nanoparticle conjugate probes into the living body, the nanoparticle conjugates probes comprising a nanoparticle; first oligonucleotides of one or more types that are bound to the nanoparticle, each type of first oligonucleotides having a sequence; and targeting conjugates of one or more types, each type of targeting conjugate comprising a targeting entity and a second oligonucleotide bound to the targeting entity and having a sequence that is complementary to a sequence of a predetermined type of the first oligonucleotides, wherein the second oligonucleotide is covalently bound to a surface of the nanoparticle, a functional group on the surface of the nanoparticle, or the first oligonucleotide; wherein the nanoparticle conjugate probes are configured to bind with one or more target analytes, wherein presence or absence of the one or more target analytes in the living body is correlated with the biological state of the living body; (ii) detecting, by a wearable device mounted on an external surface of the living body, a signal transmitted from the living body, wherein the signal includes an analyte response signal that is related to binding of the one or more target analytes with the nanoparticle conjugates; and (iii) determining a presence or absence of the one or more target analytes based on the analyte response signal.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates an example process for preparing a nanoparticle conjugate. Following hybridization, a proximity-based chemical reaction can take place to form a covalent bond between a targeting conjugate and a nanoparticle-oligonucleotide conjugate to form the nanoparticle conjugate probe.

FIG. 2 illustrates an example process for preparing a nanoparticle conjugate probes for multiplex detection of target analytes. Following hybridization, a proximity-based chemical reaction can take place to form a covalent bond between a targeting conjugate and a nanoparticle-oligonucleotide conjugate to form the nanoparticle conjugate probe.

FIG. 3 illustrates an example process for preparing nanoparticle probes for multiplex detection of target analytes.

FIG. 4 illustrates an example process for preparing a nanoparticle conjugate probe. Following hybridization, a proximity-based chemical reaction can take place to form a covalent bond between a targeting conjugate and a nanoparticle-oligonucleotide conjugate to form the nanoparticle conjugate probe. Alternatively, the proximity-based chemical reaction can be omitted.

FIG. 5 is a perspective view of an example wearable device.

FIG. 6A is a perspective top view of an example wrist-mounted device, when mounted on a wearer's wrist.

FIG. 6B is a perspective bottom view of an example wrist-mounted device shown in FIG. 6A, when mounted on a wearer's wrist.

FIG. 7A is a perspective bottom view of an example wrist-mounted device, when mounted on a wearer's wrist.

FIG. 7B is a perspective top view of an example wrist-mounted device shown in FIG. 7A, when mounted on a wearer's wrist.

FIG. 7C is a perspective view of an example wrist-mounted device shown in FIGS. 7A and 7B.

FIG. 8A is a perspective view of an example wrist-mounted device.

FIG. 8B is a perspective bottom view of an example wrist-mounted device shown in FIG. 8A.

FIG. 9 is a perspective view of an example wrist-mounted device.

FIG. 10 is a perspective view of an example wrist-mounted device.

FIG. 11 is a block diagram of an example system that includes a plurality of wearable devices in communication with a server.

FIG. 12 is a functional block diagram of an example wearable device.

FIG. 13 is a functional block diagram of an example wearable device.

FIG. 14 is a functional block diagram of an example system including a wearable device and a remote device.

FIG. 15 is a flowchart of an example method for operating a wearable device.

FIG. 16A is side partial cross-sectional view of an example wrist-mounted device, while on a human wrist.

FIG. 16B is side partial cross-sectional view of an example wrist-mounted device, while on a human wrist.

FIG. 17A is side partial cross-sectional view of an example wrist-mounted device, while on a human wrist.

FIG. 17B is side partial cross-sectional view of an example wrist-mounted device, while on a human wrist.

FIG. 18A is side partial cross-sectional view of an example wrist-mounted device, while on a human wrist.

FIG. 18B is side partial cross-sectional view of an example wrist-mounted device, while on a human wrist.

FIG. 19 is a flowchart of an example method for using a wearable device to take real-time, high-density, non-invasive, in vivo measurements of physiological parameters.

FIG. 20 is a flowchart of an example method for using a wearable device to take real-time, high-density, non-invasive, in vivo measurements of physiological parameters, in particular steps for measuring one or more analytes in blood circulating in subsurface vasculature proximate to the wearable device.

FIG. 21 is a flowchart of an example method for using a wearable device to take real-time, high-density, non-invasive, in vivo measurements of physiological parameters.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### I. Nanoparticle Conjugates

The present disclosure provides a general approach for construction of nanoparticle conjugates and libraries of such conjugates through the use of oligonucleotides to specify and facilitate desired nanoparticle functionalization for detection of target analytes or molecules. Functionalized nanoparticles are important materials for biological applications, particularly for sensing, separation, and imaging. To achieve target specificity, nanoparticles having first oligonucleotides covalently bound thereto bind via hybridization to targeting conjugates having targeting entities bound to second oligonucleotides having a sequence that is complementary to the sequence of the first oligonucleotides, wherein the second oligonucleotides include a reactive group that can covalently bind to the surface of the nanoparticle, a functional group on the nanoparticle, or to the first oligonucleotides. The resulting functionalized nanoparticle is robust and can be used to create functionalized nanoparticle libraries that achieve specificity through multivalent modification of the nanoparticles with a variety of targeting entities including, without limitation, small molecules, peptides, proteins, antibodies, antibody-fragments, aptamers etc. Any suitable reactive group can be used to covalently bind the targeting entity, which is bound to a targeting conjugate, to the surface of the nanoparticle, a functional group on the nanoparticle or on the first oligonucleotides. The functional group can include without limitation azides, alkynes, alkenes, anhydrides, amines, hydroxyls, hydroxyl carboxyls, tetrazines, thiols, and epoxy groups. The methods for producing functionalized nanoparticles or nanoparticle conjugates can be used to create libraries of robust nanoparticle conjugates that are amenable for in vitro or in vivo use for multiplex detection of target analytes.

In one aspect, a composition is provided. The composition includes a nanoparticle; first oligonucleotides of one or more types that are bound to the nanoparticle, each type of first oligonucleotides having a sequence; and targeting conjugates of one or more types, each type of targeting conjugate comprising a targeting entity and a second oligonucleotide bound to the targeting entity and having a sequence that is complementary to a sequence of a predetermined type of the first oligonucleotides, wherein the second oligonucleotide is covalently bound to a surface of the nanoparticle, a functional group on the surface of the nanoparticle or one of the first oligonucleotides. In one embodiment, the composition includes two or more types of targeting conjugates and two or more types of first oligonucleotide. In another embodiment, the composition includes a single type of first oligonucleotide and two or more types of targeting conjugates. In some embodiments, the targeting entity comprises a member of a specific binding pair.

In another aspect, a library is provided. The library includes nanoparticle conjugates of one or more types, each type of nanoparticle conjugate including nanoparticle conjugates of one or more types, each type of nanoparticle conjugate comprising: a nanoparticle; first oligonucleotides of one or more types that are bound to the nanoparticle, each type of first oligonucleotides having a sequence; and targeting conjugates of one or more types, each type of targeting conjugate comprising a targeting entity and a second oligonucleotide bound to the targeting entity and having a sequence that is complementary to a sequence of a predetermined type of the first oligonucleotides, wherein the second oligonucleotide is covalently bound to a surface of the nanoparticle, a functional group on the surface of the nanoparticle, or one of the first oligonucleotides. In one embodiment, two or more types of targeting conjugates and two or more types of first oligonucleotide are used. In another embodiment, one type of first oligonucleotide and two or more types of targeting conjugates are used. In some embodiments, the targeting entity comprises a member of a specific binding pair.

A. Nanoparticles

In general, nanoparticles contemplated include any compound or substance with a high loading capacity for an oligonucleotide as described herein, including for example and without limitation, a metal, a semiconductor, and an insulator particle composition, and a polymer (linear, branched, dendrimer (organic and inorganic)). The term “nanoparticle” refers to any particle having a diameter of less than 1000 nanometers (nm). Representative examples of nanoparticles include, without limitation, quantum dots, plasmonic nanoparticles such as gold or silver nanoparticles, upconverting nanocrystals, iron oxide nanoparticles or other superparamagnetic or magnetic particles, silica, liposomes, micelles, carbon nanotubes, doped or undoped graphene, graphene oxide, nanodiamonds, titania, alumina, and metal oxides. In some embodiments, nanoparticles can be optically or magnetically detectable. In some embodiments, intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments. Typically the nanoparticles can have a longest straight dimension (e.g., diameter) of less than 1000 nm, 900 nm, 800 nm, 700 nm, 600 nm, 500 nm, 400 nm, 300 nm, 200 nm or less. In some embodiments, the nanoparticles can have a diameter of 200 nm or less. In other embodiments, the nanoparticles have a diameter of 100 nm or less. Smaller nanoparticles, e.g. having diameters of 50 nm or less, e.g., 5 nm-30 nm, are used in some embodiments.

In one embodiment, nanoparticles are quantum dots, i.e., bright, fluorescent nanocrystals with physical dimensions small enough such that the effect of quantum confinement gives rise to unique optical and electronic properties. In certain embodiments, optically detectable nanoparticles are metal nanoparticles. Metals of use in the nanoparticles include, but are not limited to, gold, silver, iron, cobalt, zinc, cadmium, nickel, gadolinium, chromium, copper, manganese, palladium, tin, and alloys and/or oxides thereof. In some embodiments, magnetic nanoparticles are of use in accordance with the invention. “Magnetic nanoparticles” refers to magnetically responsive nanoparticles that contain one or more metals or oxides or hydroxides thereof.

In other embodiments, the nanoparticles are made from polymers or lipids See for instance, EP 2644 192; U.S. Pat. No. 8,246,968; U.S. Patent Publication No. 2013/0037977; U.S. Pat. No. 5,478,860; U.S. Patent Publ. No. 2004/0142025; International Patent Publication Nos. WO 01/05373, 2014/057432, and 2014/037498; and EP 2698066, which are incorporated by reference in their entirety.

In other embodiments, the nanoparticle comprises a bulk material that is not intrinsically fluorescent, luminescent, plasmon resonant, or magnetic. The nanoparticle comprises one or more fluorescent, luminescent, or magnetic moieties. For example, the nanoparticle may comprise QDs, fluorescent or luminescent organic molecules, or smaller nanoparticles of a magnetic material. In other embodiments, the nanoparticles are made from polymers.

In some embodiments, a nanoparticle composed in part or in whole of an organic polymer is used. A wide variety of organic polymers and methods for forming nanoparticles therefrom are known in the art. For example, nanoparticles composed at least in part of polymethylmethacrylate, polyacrylamide, polyethylene glycol, poly(vinyl chloride), carboxylated poly(vinyl chloride), or poly(vinyl chloride-co-vinyl acetate-co-vinyl alcohol) may be used. Optionally the nanoparticle comprises one or more plasticizers or additives. Co-polymers, block co-polymers, and/or grafted co-polymers can be used.

In some embodiments, the nanoparticles can be labeled with any suitable reporter label including, without limitation, fluorescent and luminescent moieties such as a variety of different organic or inorganic small molecules commonly referred to as “dyes”, “labels”, or “indicators”. Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g. Valeur, B., “Molecular Fluorescence: Principles and Applications”, John Wiley and Sons, 2002; Handbook of Fluorescent Probes and Research Products, Molecular Probes, 9th edition, 2002; and The Handbook-A Guide to Fluorescent Probes and Labeling Technologies, Invitrogen, 10^{th} edition, which are incorporated by reference in their entirety). In some embodiments, the labels can include non-fluorescent dyes or nanoparticles that can act as quenchers for fluorophores. Such nanoparticle labels may quench dynamically by distance modulation or molecular structure modulation in response to a change in the local environment or molecular recognition event.

In some embodiments, the nanoparticles can be biocompatible and/or biodegradable. As used herein, the term “biocompatible” refers to substances that are not toxic to cells or are present in levels that are not toxic to cells. In some embodiments, a substance is considered to be “biocompatible” if its addition to cells in vivo does not induce inflammation and/or other adverse effects in vivo. In other embodiments, the materials composing the nanoparticles can be generally recognized as safe (GRAS) or FDA-approved materials. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death. In general, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

In some embodiments, a nanoparticle which is biocompatible and/or biodegradable may be associated with a targeting entity and/or an agent to be delivered that is not biocompatible, is not biodegradable, or is neither biocompatible nor biodegradable. In some embodiments, a nanoparticle which is biocompatible and/or biodegradable may be associated with agent to be delivered is also biocompatible and/or biodegradable.

Nanoparticles can have a coating layer. Use of a biocompatible coating layer can be advantageous, e.g., if the nanoparticles contain materials that are toxic to cells. Suitable coating materials include, but are not limited to, natural proteins such as bovine serum albumin (BSA), biocompatible hydrophilic polymers such as polyethylene glycol (PEG) or a PEG derivative, phospholipid-(PEG), silica, lipids, polymers, carbohydrates such as dextran, and other nanoparticles, etc. Coatings may be applied or assembled in a variety of ways such as by dipping, using a layer-by-layer technique, self-assembly, conjugation, etc.

In some embodiments, the nanoparticles may optionally comprise one or more dispersion media, surfactants, release-retarding ingredients, or other pharmaceutically acceptable excipient. In some embodiments, nanoparticles may optionally comprise one or more plasticizers or additives.

In some embodiments, nanoparticles may be intrinsically magnetic nanoparticles. In some embodiments, fluorescent or luminescent nanoparticles, nanoparticles that comprise fluorescent or luminescent moieties, and plasmon resonant nanoparticles can be useful. In some embodiments, the nanoparticles have detectable optical and/or magnetic properties. In one embodiment, an optically detectable nanoparticle is one that can be detected within a living cell using optical means compatible with cell viability. In another embodiment, an optically detectable nanoparticle is one that can be detected within a living cell using optical means compatible with cell viability in a biological setting and that do not permanently compromise the integrity or viability of the cells or tissues. Optical detection is accomplished by detecting the scattering, emission, and/or absorption of light that falls within the optical region of the spectrum, i.e., that portion of the spectrum extending from approximately 400 nm to several microns. Optionally a sample containing cells is exposed to a source of electromagnetic energy. In some embodiments, absorption of electromagnetic energy (e.g. light of a given wavelength) by the nanoparticle or a component thereof is followed by the emission of light at longer wavelengths, and the emitted light is detected. In some embodiments, scattering of light by the nanoparticles is detected. In certain embodiments, light falling within the visible portion of the electromagnetic spectrum, i.e., the portion of the spectrum that is detectable by the human eye (approximately 400 nm to approximately 700 nm) is detected. In some embodiments, light that falls within the infrared or ultraviolet region of the spectrum is detected.

The optical property can be a feature of an absorption, emission, or scattering spectrum or a change in a feature of an absorption, emission, or scattering spectrum. The optical property can be a visually detectable feature such as, for example, color, apparent size, or visibility (i.e. simply whether or not the particle is visible under particular conditions). Features of a spectrum include, for example, peak wavelength or frequency (wavelength or frequency at which maximum emission, scattering intensity, extinction, absorption, etc. occurs), peak magnitude (e.g., peak emission value, peak scattering intensity, peak absorbance value, etc.), peak width at half height, or metrics derived from any of the foregoing such as ratio of peak magnitude to peak width. Certain spectra may contain multiple peaks, of which one is typically the major peak and has significantly greater intensity than the others. Each spectral peak has associated features. Typically, for any particular spectrum, spectral features such as peak wavelength or frequency, peak magnitude, peak width at half height, etc., are determined with reference to the major peak. The features of each peak, number of peaks, separation between peaks, etc., can be considered to be features of the spectrum as a whole. The foregoing features can be measured as a function of the direction of polarization of light illuminating the nanoparticles; thus polarization dependence can be measured. Features associated with hyper-Rayleigh scattering can be measured. Fluorescence detection can include detection of fluorescence modes. Luminescence detection can also be useful for optical imaging purposes. Raman scattering can also be useful as well.

In various embodiments, intrinsically fluorescent or luminescent nanoparticles, nanoparticles that comprise fluorescent or luminescent moieties, plasmon resonant nanoparticles, and magnetic nanoparticles are among the detectable nanoparticles that can be used. Such nanoparticles can have a variety of different shapes including variety of different shapes including spheres, oblate spheroids, cylinders, ovals, ellipses, shells, cubes, cuboids, cones, pyramids, rods (e.g., cylinders or elongated structures having a square or rectangular cross-section), tetrapods (nanoparticles having four leg-like appendages), triangles, prisms, etc. Nanoparticles can be also solid or hollow and can comprise one or more layers (e.g., nanoshells, nanorings, etc.). Nanoparticles may have a core/shell structure, wherein the core(s) and shell(s) can be made of different materials. Nanoparticles may comprise gradient or homogeneous alloys. Nanoparticles may be a composite made of two or more materials, of which one, more than one, or all of the materials possess magnetic properties, electrically detectable properties, and/or optically detectable properties.

In general, the nanoparticles should have dimensions small enough to prepare nano- and micro-sized composite particles. Typically the nanoparticles have a longest straight dimension (e.g., diameter) of 200 nm or less. In some embodiments, the nanoparticles have a diameter of 100 nm or less. For nano-sized composite particles, smaller nanoparticles, e.g. having diameters of 50 nm or less, e.g., 5 nm-30 nm, are useful.

B. Oligonucleotides

Each nanoparticle can have a plurality of oligonucleotides attached to it. As a result, each nanoparticle-oligonucleotide conjugate can bind to a plurality of other oligonucleotides having the complementary sequence such as a targeting conjugate discussed below. As used herein, the term “oligonucleotide” refers to short single-stranded DNA or RNA molecules of 200 or less nucleobases and includes modified forms. Likewise, the term “nucleotides” as used herein is interchangeable with modified forms as discussed herein and otherwise known in the art. In certain instances, the art uses the term “nucleobase” which embraces naturally-occurring nucleotides as well as modifications of nucleotides that can be polymerized into a molecule that functions as antisense. Herein, the terms “nucleotides” and “nucleobases” are used interchangeably to embrace the same scope unless otherwise noted. Modified bases are useful in a number of instances to minimize charge effects that a negatively charged DNA backbone may have on binding affinity (e.g., nucleic acids with methylphosphonates) or to minimize nuclease sensitivity (e.g., XNA or other nucleotide analogs). Methods of making oligonucleotides of predetermined sequences are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991), which are incorporated by reference in their entirety. Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

As used herein “nanoparticle conjugates” (also referred to as “oligonucleotide-modified nanoparticles,” “nanoparticle-oligonucleotide conjugate” and “functionalized nanoparticles”) refers to nanoparticles with one or more oligonucleotides, or modified form thereof. Any suitable length of oligonucleotides can be used to prepare the nanoparticle conjugates. In some embodiments, the oligonucleotide which modified the surface of a nanoparticle can be about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, or about 5 to about 10 nucleotides in length and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated. In some embodiments, oligonucleotides comprise from about 8 to about 80 nucleotides (i.e. from about 8 to about 80 linked nucleosides). One of ordinary skill in the art will appreciate that methods utilize compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotide in length. In some embodiments, an oligonucleotide is a DNA oligonucleotide, an RNA oligonucleotide, or a modified form of either a DNA oligonucleotide or an RNA oligonucleotide.

In various aspects, the methods include use of an oligonucleotide which is 100% complementary to the target conjugate or another oligonucleotide, i.e., a perfect match, while in other aspects, the oligonucleotide is at least (meaning greater than or equal to) about 95% complementary to the target compound over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the target compound over the length of the oligonucleotide as desired to achieve the desired degree of binding affinity of the nanoparticle conjugate to a target analyte.

The nanoparticles, the oligonucleotides or both are functionalized in order to attach the oligonucleotides to the nanoparticles to form nanoparticle conjugates. Methods for functionalizing nanoparticles and oligonucleotides are known in the art. With respect to the oligonucleotides, any suitable means for binding them to the nanoparticles can be used. Regardless of the means by which the oligonucleotides are attached to the nanoparticle, attachment in various aspects is effected through a 5′ linkage, a 3′ linkage, some type of internal linkage, or any combination of these attachments. For instance, oligonucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex., pages 109-121 (1995). See also, Mucic et al. Chem. Commun. 555-557 (1996) (describes a method of attaching 3′ thiol DNA to flat gold surfaces; this method can be used to attach oligonucleotides to nanoparticles), incorporated by reference in its entirety. The alkanethiol method can also be used to attach oligonucleotides to other metal, semiconductor and magnetic colloids and to the other nanoparticles listed above. Other functional groups for attaching oligonucleotides to solid surfaces include phosphorothioate groups. See, e.g., U.S. Pat. No. 5,472,881 for the binding of oligonucleotide-phosphorothioates to gold surfaces), substituted alkylsiloxanes (see, e.g. Burwell, Chemical Technology, 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes, all which are incorporated by reference in their entirety. Oligonucleotides terminated with a 5′ thionucleoside or a 3′ thionucleoside may also be used for attaching oligonucleotides to solid surfaces. The following references describe other methods which may be employed to attached oligonucleotides to nanoparticles: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lee et al., J. Phys. Chem., 92, 2597 (1988) (rigid phosphates on metals), all which are incorporated by reference in their entirety.

U.S. patent application Ser. Nos. 09/760,500 and 09/820,279 and International Application Nos. PCT/US01/01190 and PCT/US01/10071, incorporated by reference in their entirety, describe oligonucleotides functionalized with a cyclic disulfide. The cyclic disulfides in certain aspects have 5 or 6 atoms in their rings, including the two sulfur atoms. Suitable cyclic disulfides are available commercially or are synthesized by known procedures. Functionalization with the reduced forms of the cyclic disulfides is also contemplated.

In certain aspect, functionalized nanoparticles are contemplated which include those wherein an oligonucleotide is attached to the nanoparticle through a spacer. “Spacer” as used herein means a moiety which serves to increase distance between the nanoparticle and the functional oligonucleotide, or to increase distance between individual oligonucleotides when attached to the nanoparticle in multiple copies. Thus, spacers are contemplated being located between individual oligonucleotide in tandem, whether the oligonucleotides have the same sequence or have different sequences. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, or combinations thereof.

In certain aspects, the spacer has a moiety covalently bound to it, the moiety comprising a functional group which can bind to the nanoparticles. These are the same moieties and functional groups as described above. As a result of the binding of the spacer to the nanoparticles, the oligonucleotide is spaced away from the surface of the nanoparticles and is more accessible for hybridization with its target. In instances wherein the spacer is a polynucleotide, the length of the spacer in various embodiments at least about 10 nucleotides, 10-30 nucleotides, or even greater than 30 nucleotides. The spacer may have any sequence which does not interfere with the ability of the oligonucleotides to become bound to the nanoparticles or to the target polynucleotide. The spacers should not have sequences complementary to each other or to that of the oligonucleotides, but may be all or in part complementary to the target polynucleotide. In certain aspects, the bases of the polynucleotide spacer are all adenines, all thymines, all cytidines, all guanines, all uracils, or all some other modified base.

In another embodiment, a non-nucleotide linker of the invention comprises a basic nucleotide, polyether, polyamine, polyamide, peptide, carbohydrate, lipid, polyhydrocarbon, or other polymeric compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, the disclosures of which are all incorporated by reference herein. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine, for example at the C1 position of the sugar.

In various aspects, linkers contemplated include linear polymers (e.g., polyethylene glycol, polylysine, dextran, etc.), branched-chain polymers (see, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993, all which are incorporated by reference in their entirety); lipids; cholesterol groups (such as a steroid); or carbohydrates or oligosaccharides. Other linkers include one or more water soluble polymer attachments such as polyoxyethylene glycol, or polypropylene glycol as described U.S. Pat. Nos. 4,640,835; 4,496,689; 4,301,144; 4,670,417; 4,791,1921 and 4,179,337, all which are incorporated by reference in their entirety. Other useful polymers as linkers known in the art include monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

In still other embodiments, oligonucleotide such as poly-A or hydrophilic or amphiphilic polymers are contemplated as linkers, including, for example, amphiphiles (including oligonucleotides).

In one embodiment, a plurality of oligonucleotides may be attached to the nanoparticle. As a result, each oligonucleotide-modified composite particle can have the ability to bind to a plurality of target compounds. In some embodiments, the plurality of oligonucleotides bound to the nanoparticle may be homogenous or identical. In other embodiments, the plurality of oligonucleotides may include two or more types of oligonucleotides, each type of oligonucleotides may be different. The plurality of oligonucleotides can include about 10 to about 100,000 oligonucleotides, about 10 to about 90,000 oligonucleotides, about 10 to about 80,000 oligonucleotides, about 10 to about 70,000 oligonucleotides, about 10 to about 60,000 oligonucleotides, 10 to about 50,000 oligonucleotides, 10 to about 40,000 oligonucleotides, about 10 to about 30,000 oligonucleotides, about 10 to about 20,000 oligonucleotides, about 10 to about 10,000 oligonucleotides, and all numbers of oligonucleotides intermediate to those specifically disclosed to the extent that the oligonucleotide-modified nanoparticle is able to achieve the desired result.

In another embodiment, the oligonucleotide is bound to the nanoparticle at a surface density of at least 10 pmol/cm², at least 15 pmol/cm², at least 20 pmol/cm², at least 10 pmol/cm², at least 25 pmol/cm², at least 30 pmol/cm², at least 35 pmol/cm², at least 40 pmol/cm², at least 45 pmol/cm², at least 50 pmol/cm², at least 55 pmol/cm², at least 60 pmol/cm², at least 65 pmol/cm², at least 70 pmol/cm², or at least 75 pmol/cm².

C. Targeting Conjugate

As defined herein, a “targeting conjugate” includes a targeting entity and an oligonucleotide bound to the targeting entity. The oligonucleotide includes a sequence that is complementary to the sequence of a predetermined type of oligonucleotides bound to the nanoparticle or to the complement of the targeting sequence of an adaptor oligonucleotide (also referred to as a second oligonucleotide). Under suitable hybridization conditions, the targeting conjugate hybridizes to the nanoparticle conjugate. The oligonucleotide of the targeting conjugate include a reactive group that under suitable reaction conditions forms a covalent bond to the nanoparticle conjugate to form a nanoparticle conjugate probe. In some embodiments, the reactive group reacts directly with the surface of the nanoparticle. In other embodiments, the reactive group reacts with a functional group on the nanoparticle. In further embodiments, the reactive group reacts with the oligonucleotides bound to the nanoparticle.

In general, a “targeting entity” (also referred to as “targeting molecule”) is any entity that binds to a component (also referred to as a “target” or a “marker”) associated with a bodily fluid such as blood, an organ, tissue, cell, subcellular locale, and/or extracellular matrix component. A targeting entity may be an antibody, nucleic acid (e.g., aptamer, DNA barcodes, DNA dendrimers, DNA-zyme, RNA-zyme), peptide, glycoprotein, carbohydrate, lipid, enzyme, nanobodies, ScFv, other antibody fragments, an ionophore, small molecule recognition element, a charge carrying small molecule, etc. For example, a targeting entity can be a nucleic acid targeting entity (e.g. an aptamer) that binds to a cell type specific marker. In general, an aptamer is an oligonucleotide (e.g., DNA, RNA, or an analog or derivative thereof) that binds to a particular target, such as a polypeptide. In some embodiments, a targeting entity may be a naturally occurring or synthetic ligand for a cell surface receptor, e.g., a growth factor, hormone, LDL, transferrin, etc. A targeting entity can be an antibody, which term is intended to include antibody fragments, characteristic portions of antibodies, single chain antibodies, etc. Synthetic binding proteins such as affibodies, etc., can be used. Peptide targeting entities can be identified, e.g., using procedures such as phage display and yeast display. This widely used technique has been used to identify cell specific ligands for a variety of different cell types.

In one embodiment, the targeting entities bind to a target analyte, e.g. glucose or ion such as sodium, potassium, calcium, or chloride) in a bodily fluid such as blood, interstitium, or perspiration. In other embodiments, targeting entities bind to an organ, tissue, cell, extracellular matrix component, and/or intracellular compartment that is associated with a specific developmental stage or a specific disease state (i.e. a “target” or “marker”). In some embodiments, a target is an antigen on the surface of a cell, such as a cell surface receptor, an integrin, a transmembrane protein, an ion channel, and/or a membrane transport protein. In some embodiments, a target is an intracellular protein. In some embodiments, a target is a soluble protein, such as immunoglobulin. In some embodiments, a target is more prevalent, accessible, and/or abundant in a diseased locale (e.g. organ, tissue, cell, subcellular locale, and/or extracellular matrix component) than in a healthy locale.

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. In some embodiments, the moieties are attached to one another by one or more covalent bonds. In some embodiments, the moieties are attached to one another by a mechanism that involves specific (but non-covalent) binding (e.g. streptavidin/avidin interactions, antibody/antigen interactions, metal coordination, etc.). In some embodiments, a sufficient number of weaker interactions can provide sufficient stability for moieties to remain physically associated.

In one embodiment, the targeting agent is an antibody. As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” refers to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises at least about 200 amino acids. Antibodies to many markers are known to those of skill in the art and can be obtained commercially or readily produced by known methods such as using phage-display or yeast-display technology.

In another embodiment, the targeting agent is an aptamer. Sometimes referred to as “synthetic antibodies,” aptamers are pre-selected single-stranded oligonucleotide (e.g., DNA or RNA) or peptide molecules that bind to specific target molecules including proteins and peptides with affinities and specificities that are comparable to antibodies. These molecules can assume a variety of shapes due to their propensity to form helices and single-stranded loops with specific binding pockets, explaining their versatility in binding to diverse targets. Their specificity and characteristics are not directly determined by their primary sequence but by their tertiary structure which is analogous to the globular shape of tRNA. Aptamers have a wide range of applications including diagnostics and therapeutics and can be chemically synthesized using known techniques. Furthermore, aptamers can offer a number of advantages over traditional antibodies including avoiding the need to specifically know the precise epitopes or biomarkers themselves. Finally, aptamers are typically non-immunogenic, easy to synthesize, characterize, modify and exhibit high specificity and affinity for their target antigen.

By using a variety of selection techniques, aptamers can be selected to find targets, e.g., on a surface or inside a cell of interest or in a bodily fluid, without the need to identify the precise biomarker or epitopes themselves. In many cases, the aptamer identification process can begin with a large random pool of oligonucleotides or peptides that are systematically subjected to iterative negative and positive rounds of selection against a target, e.g., a protein molecule, to separate out low affinity or unspecific binders. The remaining aptamers in the enriched pool can be collected and propagated, e.g., PCR amplified, and used in subsequent rounds of selection. Typically anywhere from three to twenty cycles of target binding, separation, and amplification are carried out and the candidate aptamers are then characterized for binding affinity and specificity. This selection process, referred to as Systemic Evolution of Ligands by Exponential Enrichment or SELEX, is commonly used for selecting and identifying highly-targeted aptamers directed to a wide variety of targets include whole living cells. For a review of SELEX methods to screen and separate binding molecules, e.g., aptamers, from libraries of aptamers, see Stoltenburg et al. Biomolecular Engineering, 2007, Vol. 24, pp. 381-403; and Ozer et al., Molecular Therapy Nucleic Acids, 2014, Vol. 3, e183; doi:10.1038/mtna.2014.34, published on line Aug. 5, 2014, both which are incorporated by reference in their entirety. Various methods have been used for separating out the target bound and unbound aptamers including nitrocellulose filter binding, bead-based, electrophoretic, microfluidic, microarray-based, and microscopic.

The targeting entity, the oligonucleotide, or both can include linkers to bind the targeting entity and oligonucleotide to form the targeting conjugate. The term “linkers” or “linking agents” refers to a functional group that is used to link or bind two or more materials together. Linkers can be functional groups or reactive groups or may include functional or reactive groups. Functional groups include monofunctional linkers comprising a reactive group as well as multifunctional crosslinkers comprising two or more reactive groups capable of forming a bond with two or more different functional targets (e.g., labels, proteins, macromolecules, semiconductor nanocrystals, or substrate). In some preferred embodiments, the multifunctional crosslinkers are heterobifunctional crosslinkers comprising two or more different reactive groups.

Suitable reactive groups include, but are not limited to thiol (—SH), carboxylate (—COO), carboxylic acid (—COOH), amine (NH₂), hydroxyl (—OH), aldehyde (—CHO), alcohol (ROH), ketone (R₂CO), active hydrogen, ester, sulfhydryl (SH), phosphate (—PO₃), photoreactive moieties, azides, alkynes, alkenes, or tetrazines. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Other suitable reactive groups and classes of reactions include those that are well known in the art of bioconjugate chemistry. Currently favored classes of reactions available with reactive chelates are those which proceed under relatively mild conditions. These include, but are not limited to, nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March (1985) Advanced Organic Chemistry, 3rd Ed., John Wiley & Sons, New York, Hermanson (1996) Bioconjugate Techniques, Academic Press, San Diego; and Feeney et al. (1982) Modification of Proteins; Advances in Chemistry Series, Vol. 198, American Chemical Society, Washington, D.C., which are incorporated by reference in their entirety.

The targeting conjugate also includes a reactive group as described above which can be present on the targeting entity, the oligonucleotide, or both, which functions to form a covalent bond, under suitable reaction conditions with the surface of the nanoparticle, to a functional group on the nanoparticle, or to the complementary oligonucleotide that is bound to the nanoparticle and hybridized to the target conjugate.

D. Reporter Labels

In one embodiment, the nanoparticle conjugate includes a surface or a component such as the targeting conjugate, oligonucleotides, other nanoparticles, the polymeric matrix, or a linker that can be labeled with any suitable reporter labeling moiety (also referred to as “labeling moiety”, “label”, “reporter” or “reporter label”. A “labeling moiety” or “labels” as used herein, is intended to mean a chemical compound, molecule, ion, or particle that directly possesses or indirectly comes to possess a detectable signal. Representative examples of reporter labels include, without limitation, organic dyes, state dyes, environmentally-responsive absorbers that are sensitive to changes in oxygen, pH, and redox levels, fluorophores, phosphores, porphyrins, and conducting/responsive polymers. In some embodiments, the nanoparticle conjugate component can be labeled with one or more compounds or molecules such as fluorophores or auto-fluorescent or luminescent markers or non-optical contrast agents (e.g., acoustic impedance contrast, RF contrast and the like) or enzymes or enzyme substrates which may further assist in interrogating the nanoparticle conjugates in vivo. The labels can be used to indicate a conformational change of the targeting entity which can be indicative of target binding. The labeling moieties used in the current methods and compositions can be attached through any suitable means including chemical means, such as reduction, oxidation, conjugation, and condensation reactions. For example, any thiol-reactive group can be used to attach labeling moieties, e.g., a fluorophore, to a naturally occurring or engineered thiol group present in the targeting entity, e.g., aptamer or antibody. Also, for example, reactive groups present in the targeting agent can be labeled using succinimide ester derivatives of fluorophores. See Richieri, G. V. et al., J. Biol. Chem., 267: 23495-501 (1992) which is hereby incorporated by reference.

Alternatively, the targeting entity, e.g., aptamer, can be coupled to a label, e.g., a particle such as a nanoparticle, using bioorthogonal chemistries including the well-known click chemistry (i.e., the copper catalyzed alkyne azide cycloaddition reaction) which entails labeling the aptamer with an azide or alkyne group and coupling the labeled aptamer to an alkyne/azide group on the particle. Alternatively, the targeting entity, e.g., aptamer, may be labeled with an NH2 group and then coupled to —COOH group on the particle using 1-ethyl-3-[3-dimethylaminopropyl]carbodiimide hydrochloride (EDC or EDAC) cross-linking agent (commercially available from Thermo Fisher Scientific, Inc., Rockford, Ill., USA). In some embodiments, photocleavable linkers or spacers can be used to conjugate the targeting entity or nanoparticle to the reporter label. Photocleavable linkers are commercially available. See for instance Integrated DNA Technologies, Inc., Coralville, Iowa, USA; and Ambergen, Inc., Watertown, Mass., USA). Other biorthogonal reactions that can be employed for coupling the targeting agent to a label include the Staudinger ligation, copper-free click chemistry (also known as strain promoted azide alkyne cycloaddition, and tetrazine ligation. See, for instance, McKay C S and Finn M G, Click Chemistry in Complex mixtures: Bioorthogonal Bioconjugation, Chemistry & Biology, 2014; Sletten E M and Bertozzi C R. Bioorthogonal chemistry: fishing for selectivity in a sea of functionality, Angew Chem Int Ed Engl. 2009; and Greg T. Hermanson in Bioconjugate Techniques (Third Edition], 2013, Elsevier Inc., all which are incorporated by reference in its entirety.

In one embodiment, the labeling moiety can emit an optical signal. Numerous labels are known by those of skill in the art and include, but are not limited to, particles, fluorophores, haptens, enzymes and their colorimetric, fluorogenic and chemiluminescent substrates and other labels that are described in RICHARD P, HAUGLAND, MOLECULAR PROBES HANDBOOK OF FLUORESCENT PROBES AND RESEARCH PRODUCTS (9th edition, CD-ROM, (September 2002), which is herein incorporated by reference.

A fluorophore label can be any chemical moiety that exhibits an absorption maximum at or beyond 280 nm, and when covalently attached to the nanoparticle conjugate component, e.g., targeting entity, or other component retains its spectral properties. Fluorophores of the present invention include, without limitation; a pyrene (including any of the corresponding derivative compounds disclosed in U.S. Pat. No. 5,132,432, incorporated by reference), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine, a carbocyanine (including any corresponding compounds in U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; 6,664,047; 6,974,873 and 6,977,305; and publications WO 02/26891, WO 97/40104, WO 99/51702, WO 01/21624; EP 1 065 250 A1, incorporated by reference), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896, incorporated by reference), a xanthene (including any corresponding compounds disclosed in U.S. Pat. Nos. 6,162,931; 6,130,101; 6,229,055; 6,339,392; 5,451,343 and 6,716,979, incorporated by reference), an oxazine (including any corresponding compounds disclosed in U.S. Pat. No. 4,714,763, incorporated by reference) or a benzoxazine, a carbazine (including any corresponding compounds disclosed in U.S. Pat. No. 4,810,636, incorporated by reference), a phenalenone, a coumarin (including a corresponding compounds disclosed in U.S. Pat. Nos. 5,696,157; 5,459,276; 5,501,980 and 5,830,912, incorporated by reference), a benzofuran (including an corresponding compounds disclosed in U.S. Pat. Nos. 4,603,209 and 4,849,362, incorporated by reference) and benzphenalenone (including any corresponding compounds disclosed in U.S. Pat. No. 4,812,409, incorporated by reference) and derivatives thereof. As used herein, oxazines include resorufins (including any corresponding compounds disclosed in U.S. Pat. No. 5,242,805, incorporated by reference), aminooxazinones, diaminooxazines, and their benzo-substituted analogs. When the fluorophore is a xanthene, the fluorophore is optionally a fluorescein, a rhodol (including any corresponding compounds disclosed in U.S. Pat. Nos. 5,227,487 and 5,442,045, incorporated by reference), or a rhodamine (including any corresponding compounds in U.S. Pat. Nos. 5,798,276; 5,846,737 and 6,562,632, incorporated by reference). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171, incorporated by reference). Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Fluorophores for use in the present invention include, but are not limited to, xanthene (rhodol, rhodamine, fluorescein and derivatives thereof) coumarin, cyanine, pyrene, oxazine and borapolyazaindacene. Sulfonated and/or alkylated xanthenes, fluorinated xanthenes, sulfonated coumarins, fluorinated coumarins and sulfonated cyanines can be useful. The choice of the fluorophore will determine the absorption and fluorescence emission properties of the nanoparticle conjugate. Physical properties of a fluorophore label include spectral characteristics (absorption, emission and stokes shift), fluorescence intensity, lifetime, polarization and photo-bleaching rate all of which can be used to distinguish one fluorophore from another.

Binding of the nanoparticle conjugate to a target analyte may be detected with or without an interrogation signal input. The term “binding” is understood in its broadest sense to include any detectable interaction between the nanoparticle conjugate and the target analyte. For example, some nanoparticle conjugates may be functionalized with compounds or molecules, such as fluorophores or autofluorescent, luminescent or chemiluminescent markers, which generate a responsive signal with the input of a stimulus when the target diffuses into and binds on or within the nanoparticles. In other examples, the nanoparticles may produce a different responsive signal in their bound versus unbound state in response to an external stimulus, such as an electromagnetic, acoustic, optical, or mechanical energy.

In one embodiment, multiple analyte detection is possible. By immobilizing a plurality of target entities of different binding specificity to target analytes, each targeting entity associated directly or indirectly with distinct labels, e.g., different fluorophores, simultaneous multiple target analyte determinations can be made, thereby providing clinicians with deeper insight into the identification and assessment of health state and disease progression. The use of spectral filters and/or alternative light sources as the interrogation signal can be used to excite the label, e.g., fluorophores and detect light, e.g., fluorescent light, from the different labels, and thereby, determine the contribution of each fluorophore to the total fluorescent properties of the sample.

E. Linkers

Oligonucleotides, targeting conjugates, reporter labels and/or any other component (e.g, imaging agent or drug) can be attached to each other or to a surface of the nanoparticles via a reactive group or linking agent. In some embodiments, the targeting conjugate and/or the reporter can be attached to each other via a linking agent. For instance, a targeting entity and/or reporter label and nanoparticle can be conjugated via a single linking agent or multiple linking agents. For example, the targeting entity and/or reporter label and nanoparticle may be conjugated via a single multifunctional (e.g., bi-, tri-, or tetra-) linking agent or a pair of complementary linking agents. In another embodiment, the targeting agent and/or reporter label and the nanoparticle are conjugated via two, three, or more linking agents. Suitable linking agents include, but are not limited to, e.g., functional groups, affinity agents, stabilizing groups, and combinations thereof.

In certain embodiments the linking agent is or comprises a functional group. Functional groups include monofunctional linkers comprising a reactive group as well as multifunctional crosslinkers comprising two or more reactive groups capable of forming a bond with two or more different functional targets (e.g., labels, proteins, macromolecules, semiconductor nanocrystals, or substrate). In some preferred embodiments, the multifunctional crosslinkers are heterobifunctional crosslinkers comprising two or more different reactive groups.

Suitable reactive groups include, but are not limited to thiol (—SH), carboxylate (COO—), carboxylic acid (—COOH), amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties, azides, alkynes, alkenes or tetrazines. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Other suitable reactive groups and classes of reactions include those that are well known in the art of bioconjugate chemistry. For a review, see for instance, Greg T. Hermanson in Bioconjugate Techniques (Third Edition], 2013, Elsevier Inc., which is incorporated by reference in its entirety. Currently favored classes of reactions available with reactive chelates are those which proceed under relatively mild conditions. These include, but are not limited to, nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March (1985) Advanced Organic Chemistry, 3rd Ed., John Wiley & Sons, New York, Hermanson (1996) Bioconjugate Techniques, Academic Press, San Diego; and Feeney et al. (1982) Modification of Proteins; Advances in Chemistry Series, Vol. 198, American Chemical Society, Washington, D.C., which are incorporated by reference in their entirety.

In some embodiments the linking agent is a heterobifunctional crosslinker comprising two different reactive groups that form a heterocyclic ring that can interact with a peptide. For example, a heterobifunctional crosslinker such as cysteine may comprise an amine reactive group and a thiol-reactive group can interact with an aldehyde on a derivatized peptide. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. In some embodiments, an affinity agent (also referred to as a specific binding pair), e.g., agents that specifically binds to a ligand, is the linking agent. For instance, a first linking agent is bound to the semiconductor nanocrystal (nanoparticle) and a second linking agent is bound to a reporter, targeting entity, imaging or therapeutic agent. Affinity agents include receptor-ligand pairs, antibody-antigen pairs and other binding partners such as streptavidin/avidin and biotin. In one illustrative embodiment, the first linking agent is streptavidin or avidin and the second linking agent is biotin. The streptavidin or avidin is bound to the nanoparticle and a biotinylated agent (e.g., biotinylated imaging agent, biotinylated therapeutic, biotinylated antibody, etc.) is conjugated to the nanoparticle via streptavidin/avidin-biotin linkage. In some embodiments, other biotinylated radiolabel, peptides, proteins, antibodies, dyes, probes and other small molecules are attached to the streptavidin or avidin, and thus the nanoparticle.

In another embodiment, pendent functionalized linkers such as natural or modified polysaccharides as well as natural and modified nucleic or amino acids can be useful.

### II. Method for Making Nanoparticle Conjugates

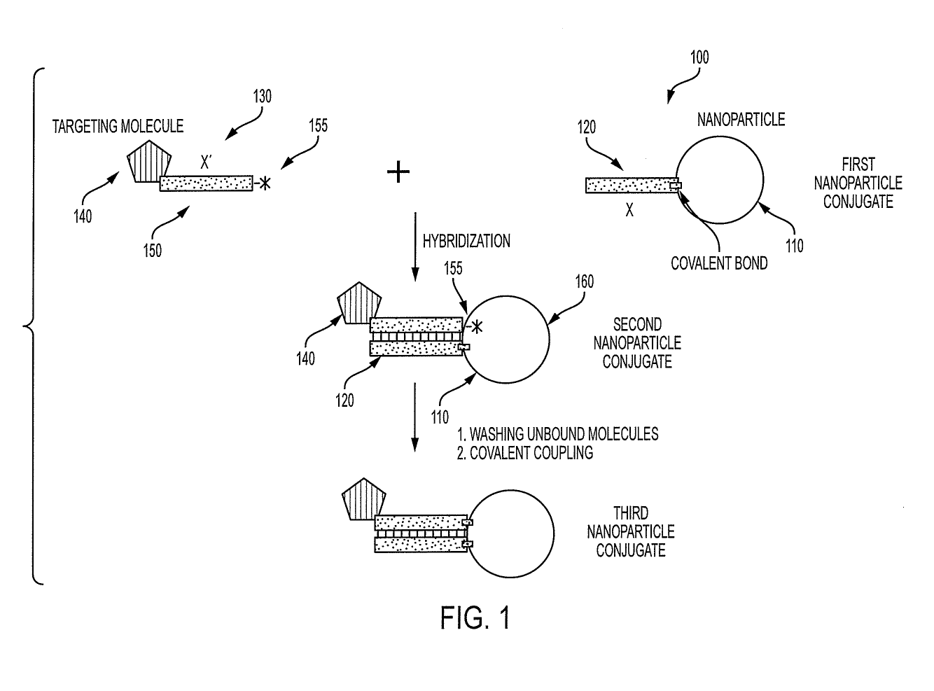

FIGS. 1-4 illustrate a number of general approaches for making the nanoparticle conjugates. These approaches employ the use of nucleic acids to specify and facilitate a desired nanoparticle-targeting molecule functionalization. In the simplest case, a nanoparticle can be covalently linked to a homogenous population of oligonucleotides (e.g., of 15-100 nucleotides in length and having a sequence X. A targeting entity or molecule (e.g, antibody) covalently coupled to a complementary nucleic acid sequence X′ (forming a targeting conjugate) can then be hybridized to sequence X. After hybridization between sequences X and X′ under suitable hybridization conditions, the targeting entity will be attached to the nanoparticle to form a probe. Further chemical reactions can be performed to form a covalent bond between the nanoparticle and the targeting conjugate to enhance the robustness of the nanoparticle. In such an approach, nanoparticles functionalized with a plurality of oligonucleotides of one type, e.g., one sequence, can be coupled to a large variety of targeting entities, provided that each targeting entity is individually functionalized with an appropriate complementary sequence (see FIG. 1). By using nucleic acids to encode the specificity of attaching targeting entities to nanoparticles, a number of advantages can be obtained including: (i) the use of universal nanoparticle conjugates whereby the rate of hybridization can be independent of the size of the targeting entity in most cases and can enable equal labeling of the nanoparticle when using a mixture of different targeting entities; (ii) functionalization reactions can be conducted in parallel in the same reaction vessel; (iii) can be potentially reversible which may allow for the release of the nanoparticle conjugate under conditions such as the presence of DNA or RNA endonucleases, introduction of nucleotide sequences recognized by a specific restriction endonuclease (restriction enzymes) such as HindII; (iv) hydrolysis in the presence of reducing agents to cleave a disulfide bond linker; enzymatic cleavage of an ester group by an esterase; (v) incorporation of specific nucleotide analogs that are sensitive to specific environmental conditions such as photocleavage by UV light; and (vi) the targeting sequence can be used as a readout, using massively parallel sequencing, to identify which targeting molecules are on a specific nanoparticle or a collection of nanoparticles.

In one aspect, a method is provided. The method includes: (a) providing a first nanoparticle conjugate of comprising a nanoparticle and a first oligonucleotides of one or more types bound to the nanoparticles, each type of first oligonucleotides having a sequence; (b) contacting the first nanoparticle conjugate with targeting conjugates of one or more types, each type of targeting conjugate comprising a targeting entity and a second oligonucleotide bound to the targeting entity, wherein the contacting occurs under suitable hybridization conditions to form a second nanoparticle conjugate, wherein the second oligonucleotide has a sequence that is complementary to a sequence of the first oligonucleotides and wherein the second oligonucleotide include a reactive group that binds to a surface of the nanoparticle, a functional group on the nanoparticle, or one of the first oligonucleotides; (c) washing the second nanoparticle conjugate; and (d) covalently reacting the reactive group of the second oligonucleotide of the second nanoparticle conjugate to the surface of the nanoparticles, a functional group on the nanoparticle, or one of the first oligonucleotides under suitable reaction conditions to form a third nanoparticle conjugate. In some embodiments, the second oligonucleotides are covalently bound to the surface or a functional group on the surface of the third nanoparticle conjugate.

As shown in FIG. 1, a nanoparticle conjugate (100) having a nanoparticle (110) bound to first oligonucleotide (120) and a targeting conjugate (130) including a targeting entity (140) linked to a second oligonucleotide (150) having a sequence that is complementary to a sequence of the first oligonucleotide (120) are provided. The targeting conjugate includes a reactive group (155). The targeting conjugate (130) and nanoparticle conjugate (100) are then contacted under suitable hybridization conditions to produce a second nanoparticle conjugate (160). The second nanoparticle conjugate (160) is then collected by any suitable means such as centrifugation or by magnetic separation (if the nanoparticles are magnetic). The second nanoparticle conjugates (160) are then washed with an suitable aqueous buffer solution such as 10 mM Tris-HCl, 150 mM NaCl, 1 mM EDTA, pH 7.5; 10 mM Tris-HCl, 150 mM LiCl, 1 mM EDTA, pH 7.5; 20 mM Tris-HCl, 1.0 M LiCl, 2 mM EDTA, pH 7.5; phosphate buffered saline; or deionized water. Thereafter, the reactive group (155) of the targeting conjugate (130) bound to the second nanoparticle conjugate (160) is then covalently bound to the surface of the nanoparticle, to a functional group on the surface of the nanoparticle, or to the first oligonucleotide bound to the nanoparticle to form a second nanoparticle conjugate which can be used as a probe for the detection of a target analyte. In the example shown in FIG. 1, the reactive group 155 reacts or binds to the surface of nanoparticle 110 or to a functional group (not shown) on the surface of the nanoparticle 110. The contacting of the oligonucleotide-modified nanoparticles with oligonucleotides takes place under conditions effective for hybridization of the oligonucleotide on the oligonucleotide-modified nanoparticle with the target sequence of the target oligonucleotide. As defined herein, “hybridization” means an interaction between two strands of nucleic acids by hydrogen bonds in accordance with the rules of Watson-Crick DNA complementarity, Hoogstein binding, or other sequence-specific binding known in the art. Hybridization can be performed under different stringency conditions known in the art. These hybridization conditions are well known in the art and can readily be optimized for the particular system employed. See, e.g., Sambrook et al., Molecular Cloning: A Laboratory Manual (2nd ed. 1989), which is incorporated by reference in its entirety. Preferably stringent hybridization conditions are employed. Under appropriate stringency conditions, hybridization between the two complementary strands could reach about 60% or above, about 70% or above, about 80% or above, about 90% or above, about 95% or above, about 96% or above, about 97% or above, about 98% or above, or about 99% or above in the reactions.

Faster hybridization can be obtained by freezing and thawing a solution containing the oligonucleotide to be detected and the oligonucleotide-modified nanoparticles. The solution may be frozen in any convenient manner, such as placing it in a dry ice-alcohol bath for a sufficient time for the solution to freeze (generally about 1 minute for 100 uL of solution). The solution must be thawed at a temperature below the thermal denaturation temperature, which can conveniently be room temperature for most combinations of oligonucleotide-modified nanoparticles and target oligonucleotides. The hybridization is complete, and the detectable change may be observed, after thawing the solution. The rate of hybridization can also be increased by warming the solution containing the target compound and the oligonucleotide-modified nanoparticle to a temperature below the dissociation temperature (Tm) for the complex formed between the oligonucleotide on oligonucleotide-modified nanoparticle and the target compound. Alternatively, rapid hybridization can be achieved by heating above the dissociation temperature (Tm) and allowing the solution to cool. The rate of hybridization can also be increased by increasing the salt concentration (e.g., from 0.1 M to 0.3 M sodium chloride).

In some embodiments, a nanoparticle-oligonucleotide conjugate can be prepared which has oligonucleotides of a single sequence attached to it. Referred to as a “universal probe”, these oligonucleotides can hybridize with target conjugates or other oligonucleotides having a sequence that is complementary to the sequence of the oligonucleotides bound to the nanoparticles. In some embodiments, the nanoparticle-oligonucleotide conjugates can be prepared having two or more types of oligonucleotides, each type having a different sequence. In such a case, these bound oligonucleotides can hybridize with target conjugates of two or more types or other oligonucleotides of two or more types, each other oligonucleotide having a sequence that is complementary to the sequence of a particular type of oligonucleotides bound to the nanoparticles. In some embodiments, the bound oligonucleotide has greater than 95%, greater than 90%, greater than 80%, greater than 75%, greater than 70%, greater than 65%, greater than 60%, greater than 55%, or greater than 50% complementary to the other oligonucleotide.

Thereafter, the resulting nanoparticle conjugates having bound target conjugates are then subjected to suitable reaction conditions such that the reactive group on the target conjugate can form a covalent bond with the surface of the nanoparticle, with a functional group on the surface of the nanoparticle, or with its complementary oligonucleotide binding partner on the nanoparticle. In a representative example, starting with a nanoparticle conjugated to the first oligonucleotides of one or more types and containing free carboxylic acids on its surface, the particles are functionalized with amine-PEG-azide (NH2-PEG-N3) using EDC and N-hydroxysulfosuccinamide (sNHS) (0.1M IVIES buffered saline, 0.9% NaCl, pH 4.7, 2 hours at room temperature). Azide activated particles are then washed in an appropriate buffer (water, PBS, etc.) with or without the addition of nonionic triblock copolymers (i.e., Pluronic) to maintain colloidal stability. The complimentary oligonucleotide can be synthesized with a 5′ or 3′ amine group using standard phosphoramidite chemistry and then treated with dibenzylcyclooctyne N-hydroxysuccinimide ester (DBCO NHS ester) to produce a cyclooctyne functionalized oligonucleotide (sodium carbonate/bicarbonate buffer, pH 9, 2-12 hours at room temperature). Oligonucleotide conjugates are desalted. After annealing the cyclooctyne functionalized oligonucleotide to its complementary strand on the surface of the particle, the complementary strand will become covalently conjugated to the particle surface through the strain-promoted azide alkyne cycloaddition reaction (i.e., copper-free click reaction) (PBS with or without the addition of nonionic triblock copolymers, 2-12 hours at room temperature). This is an example of a proximity-induced reaction. Other ways to conjugate the target conjugates to the nanoparticles can be achieved using other complementary reaction partners and the reaction conditions, e.g. such as the types of functional groups or distance between the functional groups on the particle and oligonucleotides so that they can react with each other, may need to be adjusted accordingly in order to achieve desirable yields.

In one embodiment, a library of nanoparticles can be generated. The library includes nanoparticle conjugates of one or more types, each type of nanoparticle conjugate comprising: a nanoparticle; first oligonucleotides of one or more types that are bound to the nanoparticle, each type of first oligonucleotides having a sequence; and targeting conjugates of one or more types, each type of targeting conjugate comprising a targeting entity and a second oligonucleotide bound to the targeting entity and having a sequence that is complementary to a sequence of a predetermined type of the first oligonucleotides, wherein the second oligonucleotide is covalently bound to a surface of the nanoparticle, a functional group on the surface of the nanoparticle or one of the first oligonucleotides. The library can be used to screen for a plurality of target analytes in body fluids.

In another embodiment, a method is provided which includes: (a) providing a first nanoparticle conjugate comprising a nanoparticle and a first oligonucleotide bound to the nanoparticles; (b) contacting the first nanoparticle conjugate with second oligonucleotides of one or more types, each type of second oligonucleotide comprising a targeting sequence and a sequence that is complementary to a sequence of the first oligonucleotide to produce a second nanoparticle conjugate, wherein said contacting occurs under suitable hybridization conditions; (c) extending the first oligonucleotide under suitable polymerase chain reaction conditions to produce an extended first oligonucleotide comprising a complementary sequence that is complementary to the targeting sequence of the second oligonucleotide; (d) removing the second oligonucleotide from the second nanoparticle under suitable dehybridization conditions to produce a third nanoparticle conjugate; (e) washing the third nanoparticle conjugate; (f) contacting the third nanoparticle conjugate with a targeting conjugate of one or more types, each type of targeting conjugate comprising a targeting entity and a third oligonucleotide that is bound to the targeting entity, wherein the third oligonucleotide has a predetermined sequence that is complementary to the complementary targeting sequence of the extended first oligonucleotide, wherein the third oligonucleotide has a reactive group that is capable of covalently binding to a surface of the nanoparticle, a functional group on the nanoparticle, or one of the first oligonucleotide and wherein said contacting occurs under hybridization conditions to form a fourth nanoparticle conjugate. In some embodiments, the method further comprises (f1) covalently binding the third oligonucleotide to the surface of the nanoparticle, to a functional group on the nanoparticle, or to the first oligonucleotide to form a nanoparticle conjugate probe. In other embodiments, the method further comprises: (g) extending the third oligonucleotide under suitable polymerase chain reaction conditions to produce a modified third oligonucleotide comprising a sequence that is complementary to the sequence of the first oligonucleotides; and (h) covalently reacting the reactive group of the modified third oligonucleotide with the surface of the nanoparticle or a functional group on the nanoparticle to form a nanoparticle conjugate probe. In some embodiments, the method involves a single type of second oligonucleotide and a single type of targeting conjugates. In some embodiments, the method involves two or more types of second oligonucleotides and two or more types of targeting conjugates.

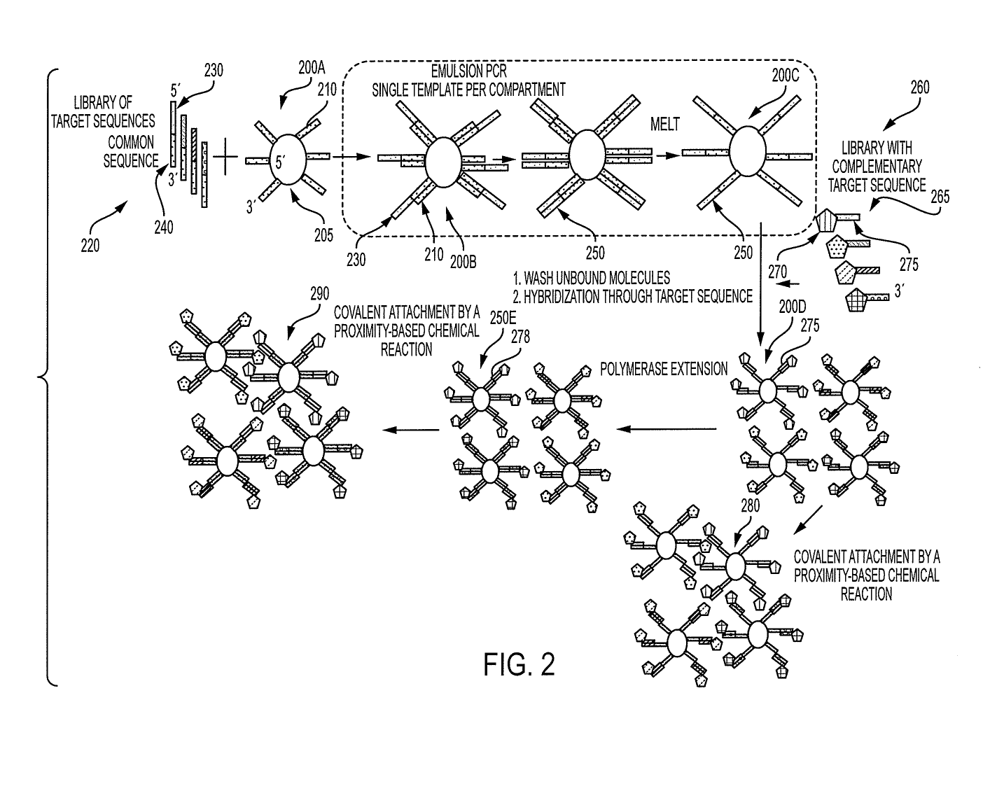

As shown in FIG. 2, a first nanoparticle conjugate (200A) having first oligonucleotides bound (210) at the 5′ end thereto bound to a nanoparticle 205 and a library of second oligonucleotides (220) of one or more types, each type of second oligonucleotide having a specific targeting molecule specific sequence (230) and a common sequence (240) that is complementary to a sequence of the first oligonucleotides (210) are provided. The second oligonucleotides and the nanoparticle conjugate are then contacted under suitable hybridization conditions to produce a second nanoparticle conjugate (200B). Each first oligonucleotide (210) bound to the second nanoparticle conjugate (200B) is then extended under suitable polymerase conditions to produce a modified first oligonucleotide (250) having a sequence that is complementary to the targeting sequence (230). The second oligonucleotides are then melted off or removed from the second nanoparticle conjugate (200B) under suitable melting temperature (Tm) or dehybridization conditions to produce a third nanoparticle conjugate (200C). The third nanoparticle conjugate (200C) is then separated by any suitable means, e.g., centrifugation, and washed with a suitable aqueous buffer solution to remove unbound molecules. Thereafter, the third nanoparticle conjugate (200C) is then contacted with a library (260) of targeting conjugates of one or more types, each type of targeting conjugates (265) including a targeting entity (270) and a third oligonucleotide (275) having a sequence that is complementary to the targeting sequence of the modified first oligonucleotide (250) under suitable hybridization conditions to produce a fourth nanoparticle conjugate (200D). The fourth nanoparticle conjugates (200D) are then collected by any suitable means such as centrifugation or by magnetic separation (if the nanoparticles are magnetic) and then washed with a suitable aqueous buffer solution such as 10 mM Tris-HCl, 150 mM NaCl, 1 mM EDTA, pH 7.5; 10 mM Tris-HCl, 150 mM LiCl, 1 mM EDTA, pH 7.5; 20 mM Tris-HCl, 1.0 M LiCl, 2 mM EDTA, pH 7.5; phosphate buffered saline; or deionized water. The fourth nanoparticle conjugates (200D) can be subjected to one or more steps to produce a robust nanoparticle probe. In one embodiment, the third oligonucleotide of the fourth nanoparticle conjugate (200D) includes a reactive group that can covalently bind to the first oligonucleotide (210) of the fourth nanoparticle conjugate (200D) under suitable reaction conditions to form the robust nanoparticle probe (280).

In an alternative embodiment as shown in FIG. 2, the fourth nanoparticle conjugates (200D) are then subjected to suitable polymerase extension conditions to extend the third oligonucleotide (275) to form fifth nanoparticle conjugate (200E) having a modified third oligonucleotide (278) having a reactive group that that covalently bind to the surface of the fifth nanoparticle conjugate (200E), to a functional group on the surface of the fifth nanoparticle conjugate (200E), or to the modified first oligonucleotide (250) of the fifth nanoparticle conjugate (200E) to form the robust nanoparticle conjugate probe (290). A representative example of this method is provided below.

In this embodiment, nanoparticles can be functionalized with a plurality of oligonucleotides having a sequence X. These “universal” nanoparticle conjugates can then be hybridized to an adaptor sequence or second oligonucleotide comprising a targeting entity specific sequence (T) and a sequence X′ complementary to sequence X. The reaction can be carried out in a multiwell plate (scale of 100 to 1000 of different nanoparticles) or in parallel using any suitable PCR based reaction, e.g., emulsion PCR. Emulsion PCR protocols are known and have been reported in the literature. See, T. Schutze et al. “A Streamlined protocol for emulsion polymerase chain reaction and subsequent purification,” Anal. Biochem. 2011, Mar. 1; 410(1):155-7, R. Williams et al. “Amplification of complex gene libraries by emulsion PCR,” Nat. Methods 3, 545-550 (2006); D. Dressman et al. “Transforming single DNA molecules into fluorescent magnetic particles for detection and enumeration of genetic variations,” PNAS, Vol. 100, no. 15, 8817-8822; Margulies et al. “Genome sequencing in microfabricated high-density picolitre reactors,” Nature 437, 376-380, all which are incorporated by reference in their entirety.

In a representative example, emulsion PCR is employed. Nanoparticles are first covalently functionalized with single-stranded DNA molecules comprised of a common sequence that serve as capture probes. The template molecules intended for clonal amplification are designed/modified with common 5′ and 3′ sequences to facilitate annealing to surface of the particles and amplification. To isolate single particles with single template reactions, PCR solution (containing appropriate oligonucleotide targeting sequence (T) with complementary adaptor sequence to the common sequence, polymerase, nucleotides, primers, and co-factors) is first added to a solution containing an appropriate number of functionalized nanoparticles (˜240 uL total volume). The mixture is allowed to sit at room temperature for at least 5 minutes to allow the beads to equilibrate with the PCR solution. To produce emulsions, 400 uL of emulsion oil (e.g. 40% w/w DC 5225C Formulation Aid (Dow), 30% w/w DC 749 Fluid (Dow), and 30% w/w Ar20 Silicone Oil (Sigma) is added on top of the 240 uL PCR-bead mixture. The emulsion is then homogenized for 5 minutes to generate emulsions small enough to contain the nanoparticles and PCR amplification mix (TissueLyser or Branson). The targeting oligonucleotide (T) is then amplified to generate multiple copies of sequence T per nanoparticle by thermal cycling the entire reaction mixture (3 minutes at 95 C, 40× (20 seconds at 98 C, 15 seconds at 60 C, 15 seconds at 72 C). This will create a library of nanoparticles where each particle is coated with multiple copies of the second oligonucleotides having sequence T. The emulsions are then ruptured (see, for example, Margulies et al. Nature 2005) and the particles are recovered and washed with annealing buffer (0.1% Tween-20, 20 mM Tris-HCl, pH 7.6, 5 mM magnesium acetate). The particles were then hybridized to targeting conjugates of interest through complementary hybridization in parallel as shown in FIG. 2.

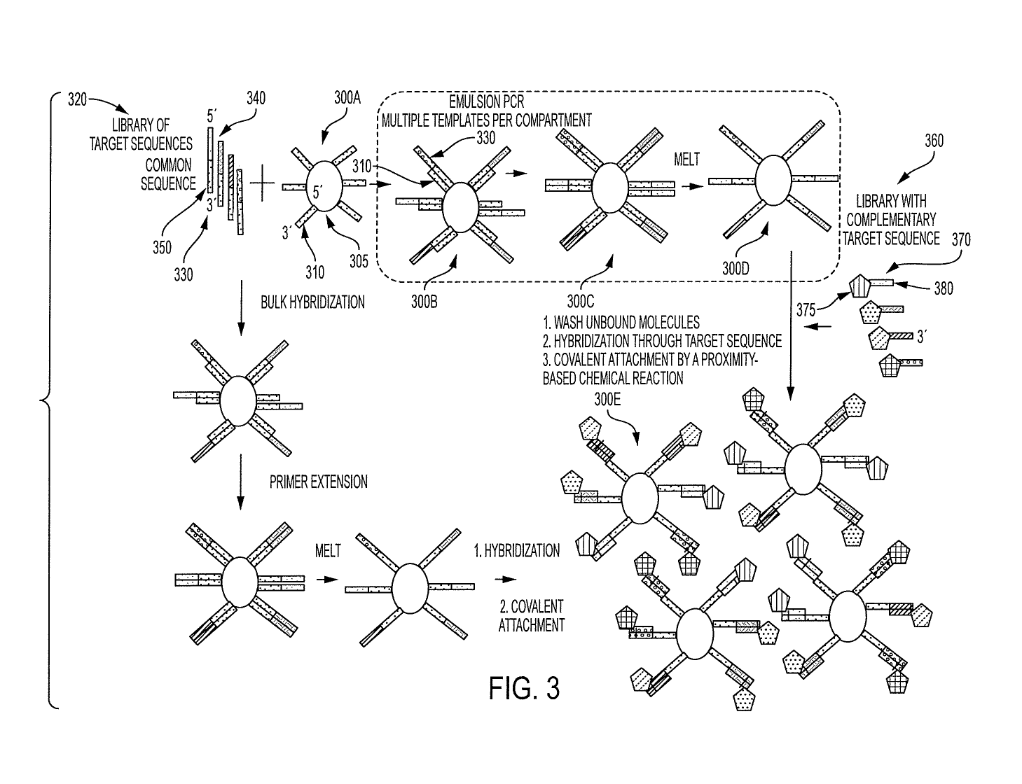

In another embodiment, as shown in FIG. 3, a first nanoparticle conjugate (300A) having one type of first oligonucleotide (310) bound at the 5′ end to nanoparticle 305 is contacted with a library (320) of second oligonucleotides (330) of one or more types, each type of second oligonucleotide having a specific targeting sequence (340) and a common sequence (350) that is complementary to a sequence of the first oligonucleotide (310) can be contacted with under suitable hybridization conditions to produce a second nanoparticle conjugate (300B), followed by polymerization extension of the first oligonucleotides (310) to produce third nanoparticle conjugate 300C. Exposure of the third nanoparticle conjugate 300C to suitable melting temperature or dehybridization conditions to remove the second oligonucleotides 330 results in a fourth nanoparticle conjugate (300D) to having a plurality of types of modified first oligonucleotides, each type having a different targeting sequences. The fourth nanoparticle conjugate (300D) is then collected, washed to remove unbound molecules, then hybridized to a library (360) of two or more types of target conjugates (370), each type of target conjugate have a target entity (375) and a third oligonucleotide (380) bound to the target entity, the third oligonucleotide 380 having a predetermined sequence that complementary to a specific targeting sequence of modified first oligonucleotides bound to the fourth nanoparticle conjugate (300D) to produce fifth nanoparticle conjugates (not shown). The fifth nanoparticle conjugates are then collected, washed, and then subjected to suitable conditions such as the reactive groups on the targeting conjugates can form a covalent bound with the surface of the nanoparticle of the fourth nanoparticle conjugates, with a functional group on the fifth nanoparticle conjugates, or the modified first oligonucleotides on the fourth nanoparticle conjugates to produce nanoparticle conjugate probes (300E). Alternatively, the nanoparticle conjugate probes (300E) can be prepared by bulk hybridization route, rather than emulsion PCR.

A representative example of this method is provided below using the emulsion PCR technique. To make nanoparticle conjugates with more complex surface reactivities, a library of different adaptor sequences (also known as second oligonucleotides) can be hybridized to a single type of nanoparticle conjugate as described above. As before, the method can be carried out in a multiwell plate or with emulsion PCR by tuning the concentration of the nucleic acids. The emulsion PCR procedure is carried out as described below with oligonucleotide templates containing a common adapter sequences. A library of target oligonucleotide sequences are synthesized with a 3′ region that is complementary to the common adaptor sequence on the nanoparticles. Selective targeting is then achieved by hybridizing the resulting nanoparticle with the library of target sequences at appropriate concentrations to ensure the desired number of targeting groups are loaded on each particle. The surface immobilized adaptors are then extended with DNA polymerase, dNTPs, and co-factors and thermal cycling (3 minutes at 95 C, 40× (20 seconds at 98 C, 15 seconds at 60 C, 15 seconds at 72 C). Templates are then melted off the particles with heat and/or 0.1N NaOH followed by extensive washing with deionized water. Conjugates either in serial or parallel as shown in FIG. 3. For preparation methods involving emulsion PCR, the sequencing of the targeting sequence for particles of interest is a relatively rapid way to determine nanoparticle identity. The targeting sequences can be used as readouts (using massively parallel sequencing) to identify which targeting molecules are on a specific nanoparticle or collection of nanoparticles.

In another embodiment, a method is provided which includes: (a) providing a first nanoparticle conjugate comprising a nanoparticle and first oligonucleotides bound to the nanoparticles; (b) contacting the first nanoparticle conjugate with a second oligonucleotide of two or more types, each type of second oligonucleotides comprising a targeting sequence and a sequence that is complementary to a sequence of the first oligonucleotide to produce a second nanoparticle conjugate, wherein said contacting occurs under suitable hybridization conditions; (c) washing the second nanoparticle conjugate; (d) contacting the second nanoparticle conjugate with targeting conjugates of one or more types, each type of targeting conjugate comprising a targeting entity and a third oligonucleotide that is bound to the targeting entity, the third oligonucleotide having a predetermined sequence that is complementary to the targeting sequence of the second oligonucleotide, wherein said contacting occurs under suitable hybridization conditions to form a third nanoparticle conjugate; and (e) ligating the third oligonucleotide to the first oligonucleotide under suitable ligation conditions to form a fourth nanoparticle conjugate. In some embodiments, the method further comprises removing or melting off the second oligonucleotide of the fourth nanoparticle conjugate under suitable melting temperature (Tm) or dehybridization conditions to form a nanoparticle conjugate probe. In other embodiments, the method further comprises covalently binding the second oligonucleotide of the fourth nanoparticle conjugate to form a nanoparticle conjugate probe. In some embodiments, the method involves a single type of second oligonucleotide and a single type of targeting conjugates are used. In other embodiments, the method involves two or more types of second oligonucleotide and two or more types of targeting conjugates.

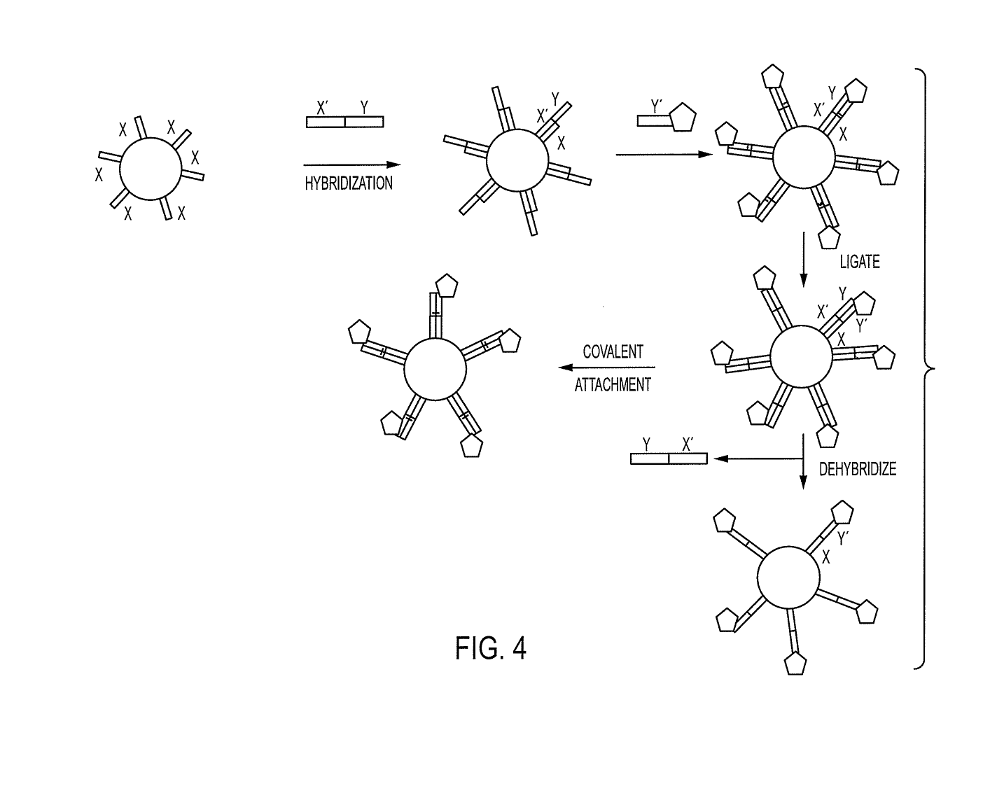

As shown in FIG. 4, a first nanoparticle conjugate (400A) having a first oligonucleotide (410) bound thereto and a second oligonucleotide (420) having a targeting molecule specific sequence (Y) and a sequence (X′) that is complementary to a sequence of the first oligonucleotide (410) are provided. The second oligonucleotide (420) the nanoparticle conjugate (400A) are then contacted under suitable hybridization conditions to produce a second nanoparticle conjugate (400B). The second nanoparticle conjugate (400B) is then collected by any suitable means such as centrifugation or by magnetic separation (if the nanoparticles are magnetic). The second nanoparticle conjugates (400B) are then washed with an suitable aqueous buffer solution such as 10 mM Tris-HCl, 150 mM NaCl, 1 mM EDTA, pH 7.5; 10 mM Tris-HCl, 150 mM LiCl, 1 mM EDTA, pH 7.5; 20 mM Tris-HCl, 1.0 M LiCl, 2 mM EDTA, pH 7.5; or deionized water. Therefore, a targeting conjugate (430) which includes a targeting entity (440) bound to a third oligonucleotide (450) having a sequence (Y′) that is complementary to the targeting molecule specific sequence (Y) of the second oligonucleotide (420) is contacted with the second nanoparticle conjugates (400B) under suitable hybridization conditions to produce a third nanoparticle conjugate (400C). The third nanoparticle conjugates (400C) are then collected by any suitable means such as centrifugation or by magnetic separation (if the nanoparticles are magnetic). The third nanoparticle conjugates (400C) are then washed with a suitable aqueous buffer solution such as 10 mM Tris-HCl, 150 mM NaCl, 1 mM EDTA, pH 7.5; 10 mM Tris-HCl, 150 mM LiCl, 1 mM EDTA, pH 7.5; 20 mM Tris-HCl, 1.0 M LiCl, 2 mM EDTA, pH 7.5; or deionized water. The third nanoparticle conjugates (400C) are then subjected to suitable ligation conditions to ligate the first oligonucleotides (410) and third oligonucleotides (450) to form the fourth nanoparticle conjugate (400D). The fourth nanoparticle conjugate (400D) can be subject to suitable melting temperature (Tm) or dehybridization conditions to remove the second oligonucleotide (420) to form the nanoparticle probe (400E). Alternatively as shown in FIG. 4, the second oligonucleotide (420) of the fourth nanoparticle conjugate (400D) can be covalently bound to the surface of the fourth nanoparticle conjugate, to a functional group of the fourth nanoparticle conjugate, or to the ligated first-third oligonucleotide under suitable reaction conditions to produce a nanoparticle conjugate probe (400F). In both instances, the targeting entity will be secured to the nanoparticle to produce a more robust nanoparticle conjugate probe (400E and 400F). A representative example of this method would be to create a second nanoparticle conjugate with four different oligonucleotide targeting sequences at density ratios of 1000:100:10:1 (A:B:C:D). These particles would then be incubated and hybridized in a solution with third oligonucleotide conjugates (A′, B′, C′, D′) where each of the third oligonucleotide conjugates is covalently linked to an antibody (A′), an aptamer (B′), a small molecule (C′), and a peptide (D)′. Ligation then produces the fourth nanoparticle conjugate, with the relative surface targeting group density defined by the ratio of the target sequences.

### II. Diagnostic System Overview

A diagnostic system can non-invasively detect and measure a plurality of physiological parameters of a person, which can include any parameters that may relate to the person's health. For example, the system could include sensors for measuring blood pressure, pulse rate, skin temperature, etc. At least some of the physiological parameters may be obtained by the system non-invasively detecting and/or measuring one or more analytes in blood circulating in subsurface vasculature. The one or more analytes could be any analytes that, when present in or absent from the blood, or present at a particular concentration or range of concentrations, may be indicative of a medical condition or health of the person. For example, the one or more analytes could include ions such as sodium potassium, calcium, and chloride, enzymes, hormones, proteins, drug metabolites, tumor cells, tumor markers or other molecules.

In an example embodiment, the system obtains at least some of the health-related information by detecting the binding or interaction of a clinically-relevant analyte to or with materials such as nanoparticle conjugates, introduced into a lumen of the subsurface vasculature that have been functionalized with a targeting entity that has a specific affinity to bind to or interact with the specific analyte such as glucose. The term “binding” is understood in its broadest sense to also include a detectable interaction between the clinically relevant analyte and the nanoparticle conjugates. The nanoparticle conjugates can be introduced into the person's blood stream by injection, ingestion, inhalation, transdermally, or in some other manner.

The nanoparticle conjugates can be functionalized by covalently or otherwise attaching or associating a targeting entity that specifically binds, undergoes cell uptake or otherwise interacts with a particular clinically-relevant target analyte with a defined affinity to the target analyte. Other compounds or molecules, such reporter labels, e.g., fluorophores or autofluorescent or luminescent markers or non-optical contrast agents (e.g. acoustic impedance contrast, RF contrast and the like), which may assist in interrogating the nanoparticles in vivo, may also be attached to the nanoparticles.

The nanoparticle conjugates includes nanoparticles having a diameter that is generally equal to or less than about 200 micrometers. In some embodiments, the nanoparticles have a diameter on the order of about 10 nanometers to 1 micrometer. In further embodiments, small nanoparticles on the order of 10-100 nanometers in diameter may be assembled to form a larger “clusters” or “assemblies on the order of 1-10 micrometers. Those of skill in the art will understand a “particle” in its broadest sense and that it may take the form of any fabricated material, a molecule, cryptophan, a virus, a phage, etc. Further, a particle may be of any shape, for example, spheres, rods, non-symmetrical shapes, etc.

In some examples, the nanoparticle conjugates include nanoparticles that can also be magnetic and can be formed from a paramagnetic, super-paramagnetic or ferromagnetic material or any other material that responds to a magnetic field. Alternatively, the nanoparticles may also be made of non-magnetic materials such as polystyrene. Where magnetic nanoparticles are used, the system may include a magnet that can direct into the portion of subsurface vasculature a magnetic field that is sufficient to manipulate aptamer-magnetic particle conjugates in a lumen of that portion of subsurface vasculature, for example, to collect or slow down in a certain area. However, measurements may be taken without localized “collection” of the nanoparticle conjugates. The system may be configured to activate the magnetic field periodically, such as at certain times of the day (e.g., every hour).

The system may further include one or more data collection systems for interrogating, in a non-invasive manner, the nanoparticle conjugates present in a lumen of the subsurface vasculature in a particular local area. In one example, the system includes a detector configured to detect a response signal transmitted from a portion of subsurface vasculature. The response signal can include both an analyte response signal, which can be related to the interaction of the one or more target analytes with the nanoparticle conjugates, and a background noise signal. For example, the nanoparticle conjugates may include a chemiluminescent marker configured to produce a response signal in the form of luminescence radiation produced in response to a chemical reaction initiated, at least in part, to the binding of the target analyte to the particle.

In some examples, the system may also include an interrogating signal source for transmitting an interrogating signal that can penetrate into a portion of subsurface vasculature, or another body system, and a detector for detecting a response signal that is transmitted from the portion of subsurface vasculature, or other body system, in response to the interrogating signal. The interrogating signal can be any kind of signal that is benign to the patient, such as electromagnetic, magnetic, optic, acoustic, thermal, mechanical, electric and results in a response signal that can be used to measure a physiological parameter or, more particularly, that can detect the binding or interaction of the clinically-relevant analyte to the nanoparticle conjugates. In one example, the interrogating signal is a radio frequency (RF) signal and the response signal is a magnetic resonance signal, such as nuclear magnetic resonance (NMR). In another example, where the nanoparticle conjugates include a fluorophore, the interrogating signal is an optical signal with a wavelength that can excite the fluorophore and penetrate the skin or other tissue and subsurface vasculature (e.g., a wavelength in the range of about 500 to about 1000 nanometers), and the response signal is fluorescence radiation from the fluorophore that can penetrate the subsurface vasculature and tissue to reach the detector. In another example, where the nanoparticle conjugates include an electrically conductive material or a magnetically lossy material, the interrogation signal may be a time-varying magnetic field or a radio frequency (RF) electromagnetic signal, with sufficient signal power to rapidly heat the nanoparticles. The response signal may be an acoustic emission from the nanoparticles, caused by rapid thermal expansion of the nanoparticles, or caused by cavitation of the liquid medium in contact with the nanoparticles. As described above, in some cases, an interrogating signal may not be necessary to produce an analyte response signal.

Additionally, the system may further include a modulation source configured to modulate the analyte response signal. The modulation source can be configured to modulate the analyte response signal differently than the background noise signal. To this end, the modulation may help to discern between the target analyte and, essentially, everything else in the body by, for example, increasing the signal-to-noise ratio. Generally, the modulation may include any spatial, temporal, spectral, thermal, magnetic, mechanical, electrical, acoustic, chemical, or electrochemical, etc. modulation technique or any combination thereof.

In some scenarios, it may also be useful to detect and distinguish both the analyte response signal—related to nanoparticle conjugates bound to or interacting with target analyte(s)—and an “unbound” particle signal—related to nanoparticle conjugates not bound to or interacting with target analyte(s). For example, in some measurement or characterization schemes, it may be useful to determine the percentage of nanoparticle conjugates introduced into the body that have bound to the target analyte. In such cases, the modulation source may be configured to modulate the analyte response signal differently than the unbound particle signal.

Data collected by the detector may be sent to a processor for analysis. The processor may be configured to non-invasively detect the one or more target analytes by differentiating the analyte response signal from the background noise signal based, at least in part, on the modulation. In some cases, the processor may further be configured to differentiate the analyte response signal from the unbound particle signal. Further, the processor may be configured to determine the concentration of a particular target analyte in the blood from, at least in part, the analyte response signal. The detection and concentration data processed by the processor may be communicated to the patient, transmitted to medical or clinical personnel, locally stored or transmitted to a remote server, the cloud, and/or any other system where the data may be stored or accessed at a later time.

The processor may be located on an external reader, which may be provided as an external body-mounted device, such as a necklace, wristwatch, eyeglasses, a mobile phone, a handheld or personal computing device or some combination thereof. Data collected by the detector may be transmitted to the external reader via a communication interface. Control electronics can wirelessly communicate the data to the external reader by modifying the impedance of an antenna in communication with the detector so as to characteristically modify the backscatter from the antenna. In some examples, the external reader can operate to intermittently interrogate the detector to provide a reading by radiating sufficient radiation to power the detector to obtain a measurement and communicate the result. In this way, the external reader can acquire a series of analyte identification and concentration measurements over time without continuously powering the detector and/or processor. The processor may also be provided at another location distal to the detector, and the detector data is communicated to the processor via a wired connection, a memory card, a USB device or other known method. Alternatively, the processor may be located proximal to the detector and may be configured to locally analyze the data that it collects and then transmit the results of the analysis to an external reader or server.

The external reader may include a user interface, or may further transmit the collected data to a device with a user interface that can indicate the results of the data analysis. In this way, the person wearing, holding or viewing the device can be made aware of the analysis and/or potential medical conditions. The external reader may also be configured to produce an auditory or tactile (vibration) response to alert the patient of a medical condition. Further, the external reader may also be configured to receive information from the patient regarding his/her health state, wellness state, activity state, nutrition intake and the like, as additional input information to the processor. For example, the user may input a health or wellness state, such as, experiencing migraine symptoms, jittery, racing heart, upset stomach, feeling tired, activity state including types and duration of physical activity nutrition intake including meal timing and composition, and other parameters including body weight, medication intake, quality of sleep, stress level, personal care products used, environmental conditions, social activity, etc. Further, the reader may also receive signals from one or more other detectors, such as a pedometer, heart rate sensor, blood pressure sensor, blood oxygen saturation level, body temperature, GPS or other location or positioning sensors, microphone, light sensor, etc.

The system may be configured to obtain data during pre-set measurement periods or in response to a prompt. For example, the system may be configured to operate the detector and collect data once an hour. In other examples, the system may be configured to operate the detector in response to a prompt, such as a manual input by the patient or a physician. The system may also be configured to obtain data in response to an internal or external event or combination of events, such as during or after physical activity, at rest, at high pulse rates, high or low blood pressures, cold or hot weather conditions, etc. In other examples, the system could operate the detector more frequently or less frequently, or the system could measure some analytes more frequently than others.

Data collected by the system may be used to notify the patient of, as described above, analyte levels or of an existing or imminent medical emergency. In some examples, the data may be used to develop an individual baseline profile for the patient. The baseline profile may include patterns for how one or more of the patient's analyte levels typically change over time, such as during the course of a day, a week, or a month, or in response to consumption of a particular type of food/drug. The baseline profile, in essence, may establish “normal” levels of the measured analytes for the patient. Additional data, collected over additional measurement periods, may be compared to the baseline profile. If the additional data is consistent with the patterns embodied in the baseline profile, it may be determined that the patient's condition has not changed. On the other hand, if the additional data deviates from the patterns embodied in the baseline profile, it may be determined that the patient's condition has changed. The change in condition could, for example, indicate that the patient has developed a disease, disorder, or other adverse medical condition or may be at risk for a severe medical condition in the near future. Further, the change in condition could further indicate a change in the patient's eating habits, either positively or negatively, which could be of interest to medical personnel. Further, the patient's baseline and deviations from the baseline can be compared to baseline and deviation data collected from a population of wearers of the devices.

When a change in condition is detected, a clinical protocol may be consulted to generate one or more recommendations that are appropriate for the patient's change in condition. For example, it may be recommended that the patient inject himself/herself with insulin, change his/her diet, take a particular medication or supplement, schedule an appointment with a medical professional, get a specific medical test, go to the hospital to seek immediate medical attention, abstain from certain activities, etc. The clinical protocol may be developed based, at least in part, on correlations between analyte concentration and health state derived by the server, any known health information or medical history of the patient, and/or on recognized standards of care in the medical field. The one or more recommendations may then be transmitted to the external reader for communication to the user via the user interface.

Correlations may be derived between the analyte concentration(s) measured by the system and the health state reported by the patient. For example, analysis of the analyte data and the health state data may reveal that the patient has not responded to chemotherapy when an analyte reaches a certain concentration. This correlation data may be used to generate recommendations for the patient, or to develop a clinical protocol. Blood analysis may be complemented with other physiological measurements such as blood pressure, heart rate, body temperature etc., in order to add to or enhance these correlations.

Further, data collected from a plurality of patients, including both the analyte measurements and the indications of health state, may be used to develop one or more clinical protocols used by the server to generate recommendations and/or used by medical professionals to provide medical care and advice to their patients. This data may further be used to recognize correlations between blood analytes and health conditions among the population. Health professionals may further use this data to diagnose and prevent illness and disease, prevent serious clinical events in the population, and to update clinical protocols, courses of treatment, and the standard of care.

The above described system may be implemented as a device. In one embodiment, the device is a wearable device. The term “wearable device,” as used in this disclosure, refers to any device that is capable of being worn at, on or in proximity to a body surface, such as a wrist, ankle, waist, chest, ear, eye or other body part. In order to take in vivo measurements in a non-invasive manner from outside of the body, the wearable device may be positioned on a portion of the body where subsurface vasculature is easily observable, the qualification of which will depend on the type of detection system used. The device may be placed in close proximity to the skin or tissue, but need not be touching or in intimate contact therewith. A mount, such as a belt, wristband, ankle band, headband, etc. can be provided to mount the device at, on or in proximity to the body surface. The mount may prevent the wearable device from moving relative to the body to reduce measurement error and noise. Further, the mount may be an adhesive substrate for adhering the wearable device to the body of a wearer. The detector, modulation source, interrogation signal source (if applicable) and, in some examples, the processor, may be provided on the wearable device. In other embodiments, the above described system may be implemented as a stationary measurement device to which a user must be brought into contact or proximity with or as a device that may be temporarily placed or held against a body surface during one or more measurement periods.

It should be understood that the above embodiments, and other embodiments described herein, are provided for explanatory purposes, and are not intended to be limiting.

### III. Example Wearable Devices

In some examples, the wearable devices described herein obtain at least some of the health-related information by detecting the binding of a clinically-relevant analyte, such as a tumor marker, to the nanoparticle conjugates. The nanoparticle conjugates can be introduced into the person's blood stream by injection, ingestion, inhalation, transdermally, or in some other suitable manner.

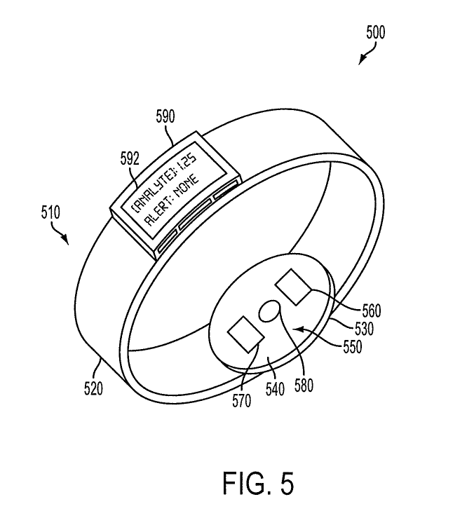

A wearable can automatically measure a plurality of physiological parameters of a person wearing the device. The term “wearable device,” as used in this disclosure, refers to any device that is capable of being worn at, on or in proximity to a body surface, such as a wrist, ankle, waist, chest, or other body part. In order to take in vivo measurements in a non-invasive manner from outside of the body, the wearable device may be positioned on a portion of the body where subsurface vasculature is easily observable, the qualification of which will depend on the type of detection system used. The device may be placed in close proximity to the skin or tissue, but need not be touching or in intimate contact therewith. A mount 510, such as a belt, wristband, ankle band, etc. can be provided to mount the device at, on or in proximity to the body surface. The mount 510 may prevent the wearable device from moving relative to the body to reduce measurement error and noise. In one example, shown in FIG. 5, the mount 510, may take the form of a strap or band 520 that can be worn around a part of the body. Further, the mount 510 may be an adhesive substrate for adhering the wearable device 500 to the body of a wearer.

A measurement platform 530 is disposed on the mount 510 such that it can be positioned on the body where subsurface vasculature is easily observable. An inner face 540 of the measurement platform is intended to be mounted facing to the body surface. The measurement platform 530 may house a data collection system 550, which may include at least one detector 560 for detecting at least one physiological parameter. The at least one physiological parameter could be any parameter that may relate to the health of the person wearing the wearable device. For example, the detector 560 could be configured to measure blood pressure, pulse rate, respiration rate, skin temperature, etc. At least one of the detectors 560 is configured to non-invasively measure one or more analytes in blood circulating in subsurface vasculature proximate to the wearable device. In a non-exhaustive list, detector 560 may include any one of an optical (e.g., CMOS, CCD, photodiode), acoustic (e.g., piezoelectric, piezoceramic), electrochemical (voltage, impedance), thermal, mechanical (e.g., pressure, strain), magnetic, or electromagnetic (e.g., magnetic resonance) sensor. The components of the data collection system 550 may be miniaturized so that the wearable device may be worn on the body without significantly interfering with the wearer's usual activities.

In some examples, the data collection system 550 further includes a signal source 570 for transmitting an interrogating signal that can penetrate the wearer's skin into the portion of subsurface vasculature, for example, into a lumen of the subsurface vasculature. The interrogating signal can be any kind of signal that is benign to the wearer, such as electromagnetic, magnetic, optic, acoustic, thermal, mechanical, and results in a response signal that can be used to measure a physiological parameter or, more particularly, that can detect the binding of the clinically-relevant analyte to the nanoparticle conjugates. In one example, the interrogating signal is an electromagnetic pulse (e.g., a radio frequency (RF) pulse) and the response signal is a magnetic resonance signal, such as nuclear magnetic resonance (NMR). In another example, the interrogating signal is a time-varying magnetic field, and the response signal is an externally-detectable physical motion due to the time-varying magnetic field. The time-varying magnetic field modulates the nanoparticles by physical motion in a manner different from the background, making them easier to detect. In a further example, the interrogating signal is an electromagnetic radiation signal. In particular, the interrogating signal may be electromagnetic radiation having a wavelength between about 500 nanometers and about 1600 nanometers. The interrogating signal may, more particularly, comprise electromagnetic radiation having a wavelength between about 500 nanometers and about 1000 nanometers. In some examples, the nanoparticle conjugates include a fluorophore. The interrogating signal may therefore be an electromagnetic radiation signal with a wavelength that can excite the fluorophore and penetrate the skin or other tissue and subsurface vasculature (e.g., a wavelength in the range of about 500 to about 1000 nanometers), and the response signal is fluorescence radiation from the fluorophore that can penetrate the subsurface vasculature and tissue to reach the detector.

In some cases, an interrogating signal is not necessary to measure one or more of the physiological parameters and, therefore, the wearable device 500 may not include a signal source 570. For example, the nanoparticle conjugates include an autofluorescent or luminescent marker, such as a fluorophore, that will automatically emit a response signal indicative of the binding of the clinically-relevant analyte to the nanoparticle conjugates, without the need for an interrogating signal or other external stimulus. In some examples, the nanoparticle conjugates may include a chemiluminescent marker configured to produce a response signal in the form of luminescence radiation produced in response to a chemical reaction initiated, at least in part, to the binding of the target analyte to the particle.

A collection magnet 580 may also be included in the data collection system 550. In such embodiments, the nanoparticle conjugates may also be made of or be functionalized with magnetic materials, such as ferromagnetic, paramagnetic, super-paramagnetic, or any other material that responds to a magnetic field. The collection magnet 580 is configured to direct a magnetic field into the portion of subsurface vasculature that is sufficient to cause the magnetic nanoparticle conjugates to collect in a lumen of that portion of subsurface vasculature. The magnet may be an electromagnet that may be turned on during measurement periods and turned off when a measurement period is complete so as to allow the magnetic nanoparticles to disperse through the vasculature.

The wearable device 500 may also include a user interface 590 via which the wearer of the device may receive one or more recommendations or alerts generated either from a remote server or other remote computing device, or from a processor within the device. The alerts could be any indication that can be noticed by the person wearing the wearable device. For example, the alert could include a visual component (e.g., textual or graphical information on a display), an auditory component (e.g., an alarm sound), and/or tactile component (e.g., a vibration). Further, the user interface 590 may include a display 592 where a visual indication of the alert or recommendation may be displayed. The display 592 may further be configured to provide an indication of the measured physiological parameters, for instance, the concentrations of certain blood analytes being measured.

The wearable device may, in some cases, also include a modulation source. The signal-to-noise ratio (SNR) in an analyte detection system, such as any of those described above, may be increased by modulating the analyte response signal transmitted from the subsurface vasculature (or other body system) with respect to the background signal and, in some cases, an unbound particle response signal. Such modulation can increase the system's sensitivity and ability to discern between target analytes present in the blood or other bodily fluids, versus other analytes, nanoparticles, cells, molecules, blood components, bone and tissues, etc. This can be particularly valuable with some methods of analyte characterization, such as optical methods, or where the target analytes are rare in the blood or are of a relatively small size and with fluorescence detection techniques, which can often suffer from low resolution because other tissues, cells, and molecules in the body may have some inherent fluorescent properties, creating a high level of background noise.

The modulation source may apply a modulation, configured to modulate the response signal, to the portion of the body. Specifically, the modulation source may be configured to modulate the analyte response signal differently from a background signal. The background signal may include any signal transmitted from something other than what the system is monitoring, i.e., the target analyte(s). In some examples, the background signal may be generated by other molecules, cells, or nanoparticles in the blood or other bodily fluids; tissue, such as skin, veins, muscle, etc.; bone; or other objects present in the wearer's body. A background signal may be generated by excitation of these objects from the interrogating signal, such as by generating an autofluorescence signal, or due to some inherent property of these objects, such as, chemiluminescence, etc.

In some examples, the modulation source may be configured to modulate the analyte response signal (transmitted from bound nanoparticles) differently than the unbound particle signal (transmitted from nanoparticles that are not bound or otherwise interacting with the target analyte(s)), such that the analyte response signal may be differentiated from the unbound particle signal. Such differentiation may be used to determine the number or percentage of nanoparticles bound to or interacting with the target analyte(s), which may be used to determine a concentration of the target analyte(s) in the blood or other bodily fluid, to determine if and to what extent the nanoparticles are being cleared from the body, etc.

The modulation source may include any means for modulating the response signal. In some cases, the analyte response signal may be modulated differently than the background signal, and in other cases the analyte response signal may be modulated differently than the unbound particle signal, or both. For example, the modulation source may be configured to alter the spatial, optical magnetic, electric, acoustic, and/or physical properties of the bound nanoparticles. The modulation source may be a physical construct or it may be a signal or energy applied to the body, or a combination thereof. Accordingly, the modulation may include spatial, temporal, spectral, thermal, magnetic, optical, mechanical, electrical, acoustic, chemical, or electrochemical type of modulation or any combination thereof.

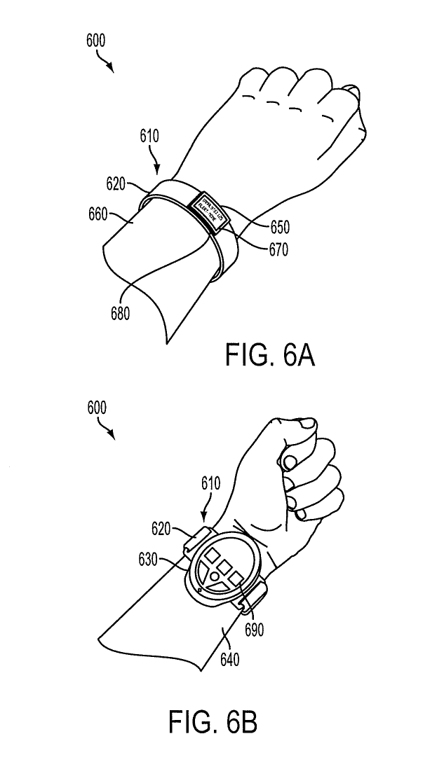

In one example, the wearable device is provided as a wrist-mounted device, as shown in FIGS. 6A, 6B, 7A-7C, 8A, 9B, and 10. The wrist-mounted device may be mounted to the wrist of a living subject with a wristband or cuff, similar to a watch or bracelet. As shown in FIGS. 6A and 6B, the wrist mounted device 600 may include a mount 610 in the form of a wristband 620, a measurement platform 630 positioned on the anterior side 640 of the wearer's wrist, and a user interface 650 positioned on the posterior side 660 of the wearer's wrist. The wearer of the device may receive, via the user interface 650, one or more recommendations or alerts generated either from a remote server or other remote computing device, or alerts from the measurement platform. Such a configuration may be perceived as natural for the wearer of the device in that it is common for the posterior side 660 of the wrist to be observed, such as the act of checking a wrist-watch. Accordingly, the wearer may easily view a display 670 on the user interface. Further, the measurement platform 630 may be located on the anterior side 640 of the wearer's wrist where the subsurface vasculature may be readily observable. However, other configurations are contemplated.

The display 670 may be configured to display a visual indication of the alert or recommendation and/or an indication of the measured physiological parameters, for instance, the concentrations of certain blood analytes being measured. Further, the user interface 650 may include one or more buttons 680 for accepting inputs from the wearer. For example, the buttons 680 may be configured to change the text or other information visible on the display 670. As shown in FIG. 6B, measurement platform 630 may also include one or more buttons 690 for accepting inputs from the wearer. The buttons 690 may be configured to accept inputs for controlling aspects of the data collection system, such as initiating a measurement period, or inputs indicating the wearer's current health state (i.e., normal, migraine, shortness of breath, heart attack, fever, “flu-like” symptoms, food poisoning, etc.).

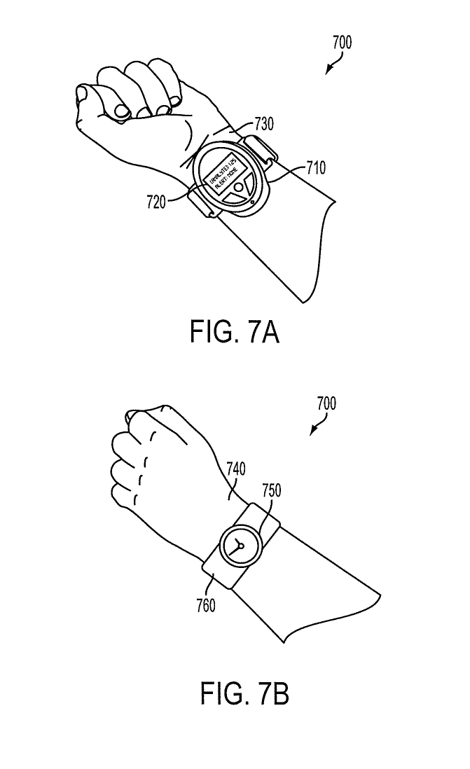

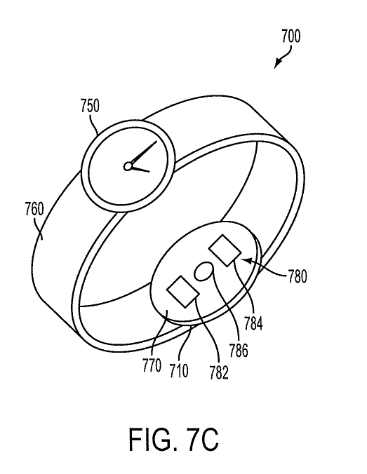

In another example wrist-mounted device 700, shown in FIGS. 7A-7C, the measurement platform 710 and user interface 720 are both provided on the same side of the wearer's wrist, in particular, the anterior side 730 of the wrist. On the posterior side 740, a watch face 750 may be disposed on the strap 760. While an analog watch is depicted in FIG. 7B, one of ordinary skill in the art will recognize that any type of clock may be provided, such as a digital clock.

As can be seen in FIG. 7C, the inner face 770 of the measurement platform 710 is intended to be worn proximate to the wearer's body. A data collection system 780 housed on the measurement platform 710 may include a detector 782, a signal source 784 and a collection magnet 786. As described above, the signal source 784 and the collection magnet 786 may not be provided in all embodiments of the wearable device.

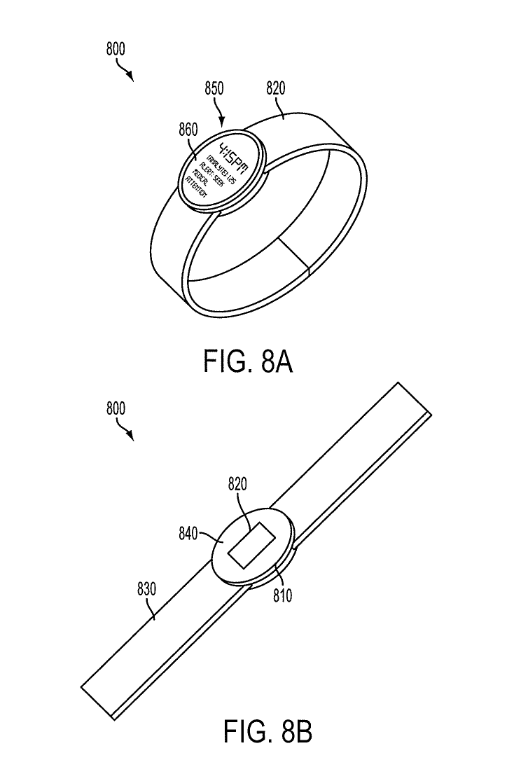

In a further example shown in FIGS. 8A and 8B, a wrist mounted device 800 includes a measurement platform 810, which includes a data collection system 820, disposed on a strap 830. Inner face 840 of measurement platform may be positioned proximate to a body surface so that data collection system 820 may interrogate the subsurface vasculature of the wearer's wrist. A user interface 850 with a display 860 may be positioned facing outward from the measurement platform 810. As described above in connection with other embodiments, user interface 850 may be configured to display data collected from the data collection system 820, including the concentration of one or more measured analytes, and one or more alerts generated by a remote server or other remote computing device, or a processor located on the measurement platform. The user interface 820 may also be configured to display the time of day, date, or other information that may be relevant to the wearer.

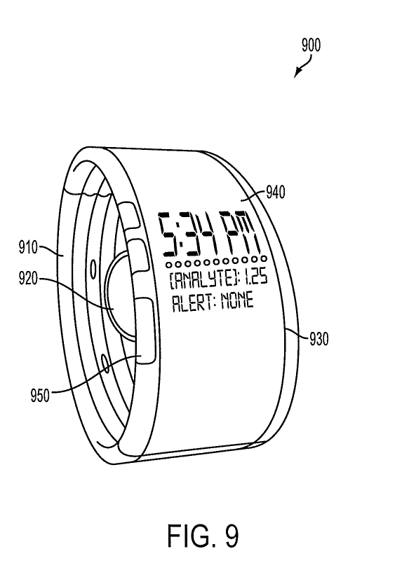

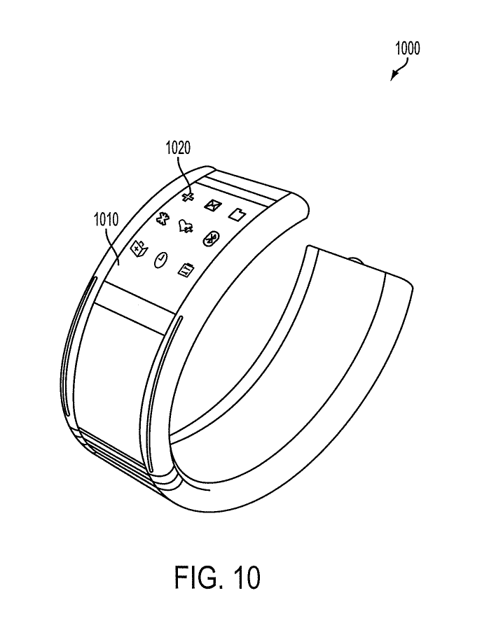

As shown in FIG. 9, in a further embodiment, wrist-mounted device 900 may be provided on a cuff 910. Similar to the previously discussed embodiments, device 900 includes a measurement platform 920 and a user interface 930, which may include a display 940 and one or more buttons 950. The display 940 may further be a touch-screen display configured to accept one or more input by the wearer. For example, as shown in FIG. 10, display 1010 may be a touch-screen configured to display one or more virtual buttons 1020 for accepting one or more inputs for controlling certain functions or aspects of the device 1000, or inputs of information by the user, such as current health state.

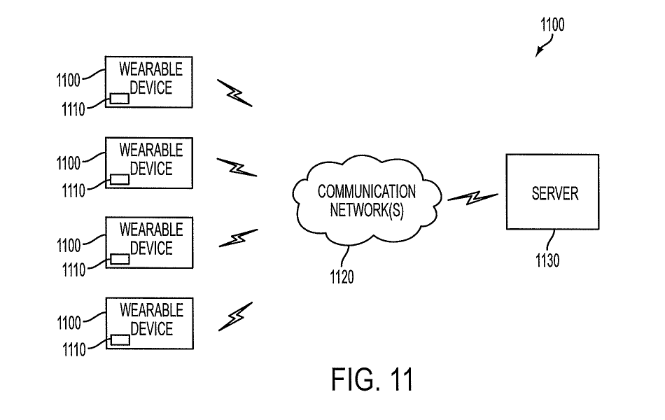

FIG. 11 is a simplified schematic of a system including one or more wearable devices 1100. The one or more wearable devices 1100 may be configured to transmit data via a communication interface 1110 over one or more communication networks 1120 to a remote server 1130. In one embodiment, the communication interface 1110 includes a wireless transceiver for sending and receiving communications to and from the server 1130. In further embodiments, the communication interface 1110 may include any means for the transfer of data, including both wired and wireless communications. For example, the communication interface may include a universal serial bus (USB) interface or a secure digital (SD) card interface. Communication networks 1120 may be any one of may be one of: a plain old telephone service (POTS) network, a cellular network, a fiber network and a data network. The server 1130 may include any type of remote computing device or remote cloud computing network. Further, communication network 1120 may include one or more intermediaries, including, for example wherein the wearable device 1100 transmits data to a mobile phone or other personal computing device, which in turn transmits the data to the server 1130.

In addition to receiving communications from the wearable device 1100, such as collected physiological parameter data and data regarding health state as input by the user, the server may also be configured to gather and/or receive either from the wearable device 1100 or from some other source, information regarding a wearer's overall medical history, environmental factors and geographical data. For example, a user account may be established on the server for every wearer that contains the wearer's medical history. Moreover, in some examples, the server 1130 may be configured to regularly receive information from sources of environmental data, such as viral illness or food poisoning outbreak data from the Centers for Disease Control (CDC) and weather, pollution and allergen data from the National Weather Service. Further, the server may be configured to receive data regarding a wearer's health state from a hospital or physician. Such information may be used in the server's decision-making process, such as recognizing correlations and in generating clinical protocols.

Additionally, the server may be configured to gather and/or receive the date, time of day and geographical location of each wearer of the device during each measurement period. Such information may be used to detect and monitor spatial and temporal spreading of diseases. As such, the wearable device may be configured to determine and/or provide an indication of its own location. For example, a wearable device may include a GPS system so that it can include GPS location information (e.g., GPS coordinates) in a communication to the server. As another example, a wearable device may use a technique that involves triangulation (e.g., between base stations in a cellular network) to determine its location. Other location-determination techniques are also possible.

The server may also be configured to make determinations regarding the efficacy of a drug or other treatment based on information regarding the drugs or other treatments received by a wearer of the device and, at least in part, the physiological parameter data and the indicated health state of the user. From this information, the server may be configured to derive an indication of the effectiveness of the drug or treatment. For example, if a drug is intended to treat nausea and the wearer of the device does not indicate that he or she is experiencing nausea after beginning a course of treatment with the drug, the server may be configured to derive an indication that the drug is effective for that wearer. In another example, a wearable device may be configured to measure tumor marker concentrations. If a wearer is prescribed a drug intended to treat cancer, but the server receives data from the wearable device indicating that the wearer's tumor marker concentration has been increasing over a certain number of measurement periods, the server may be configured to derive an indication that the drug is not effective for its intended purpose for this wearer.

Further, some embodiments of the system may include privacy controls which may be automatically implemented or controlled by the wearer of the device. For example, where a wearer's collected physiological parameter data and health state data are uploaded to a cloud computing network for trend analysis by a clinician, the data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined.

Additionally or alternatively, wearers of a device may be provided with an opportunity to control whether or how the device collects information about the wearer (e.g., information about a user's medical history, social actions or activities, profession, a user's preferences, or a user's current location), or to control how such information may be used. Thus, the wearer may have control over how information is collected about him or her and used by a clinician or physician or other user of the data. For example, a wearer may elect that data, such as health state and physiological parameters, collected from his or her device may only be used for generating an individual baseline and recommendations in response to collection and comparison of his or her own data and may not be used in generating a population baseline or for use in population correlation studies.

### IV. Example Electronics Platform for a Wearable Device

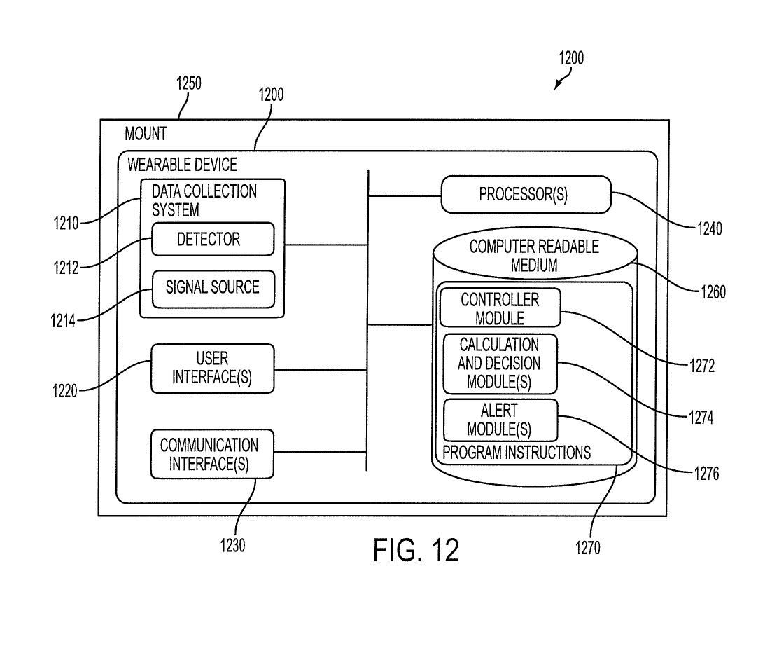

FIG. 12 is a simplified block diagram illustrating the components of a wearable device 1200, according to an example embodiment. Wearable device 900 may take the form of or be similar to one of the wrist-mounted devices 600, 700, 800, 900, 1000, shown in FIGS. 6A-B, 7A-7C, 8A-8C, 9 and 10. However, wearable device 1200 may also take other forms, such as an ankle, waist, or chest-mounted device.

In particular, FIG. 12 shows an example of a wearable device 1200 having a data collection system 1210, a user interface 1220, communication platform 1230 for transmitting data to a server, and processor(s) 1240. The components of the wearable device 1200 may be disposed on a mount 1250 for mounting the device to an external body surface where a portion of subsurface vasculature is readily observable.

Processor 1240 may be a general-purpose processor or a special purpose processor (e.g., digital signal processors, application specific integrated circuits, etc.). The one or more processors 1240 can be configured to execute computer-readable program instructions 1270 that are stored in the computer readable medium 1260 and are executable to provide the functionality of a wearable device 1200 described herein.

The computer readable medium 1260 may include or take the form of one or more non-transitory, computer-readable storage media that can be read or accessed by at least one processor 1240. The one or more computer-readable storage media can include volatile and/or non-volatile storage components, such as optical, magnetic, organic or other memory or disc storage, which can be integrated in whole or in part with at least one of the one or more processors 1240. In some embodiments, the computer readable medium 1260 can be implemented using a single physical device (e.g., one optical, magnetic, organic or other memory or disc storage unit), while in other embodiments, the computer readable medium 1260 can be implemented using two or more physical devices.

Data collection system 1210 includes a detector 1212 and, in some embodiments, a signal source 1214. As described above, detector 1212 may include any detector capable of detecting at least one physiological parameter, which could include any parameters that may relate to the health of the person wearing the wearable device. For example, the detector 1212 could be configured to measure blood pressure, pulse rate, skin temperature, etc. At least one of the detectors 1212 is configured to non-invasively measure one or more analytes in blood circulating in subsurface vasculature proximate to the wearable device. In some examples, detector 1212 may include one or more of an optical (e.g., CMOS, CCD, photodiode), acoustic (e.g., piezoelectric, piezoceramic), electrochemical (voltage, impedance), thermal, mechanical (e.g., pressure, strain), magnetic, or electromagnetic (e.g., magnetic resonance) sensor.

In some examples, the data collection system 1210 further includes a signal source 1214 for transmitting an interrogating signal that can penetrate the wearer's skin into the portion of subsurface vasculature. In general, signal source 1214 will generate an interrogation signal that will produce a responsive signal that can be detected by one or more of the detectors 1212. The interrogating signal can be any kind of signal that is benign to the wearer, such as electromagnetic, magnetic, optic, acoustic, thermal, mechanical, and results in a response signal that can be used to measure a physiological parameter or, more particularly, that can detect the binding of the clinically-relevant analyte to the nanoparticle conjugates. In one example, the interrogating signal is an electromagnetic pulse (e.g., a radio frequency (RF) pulse) and the response signal is a magnetic resonance signal, such as nuclear magnetic resonance (NMR). In another example, the interrogating signal is a time-varying magnetic field, and the response signal is an externally-detectable physical motion due to the time-varying magnetic field. The time-varying magnetic field modulates the nanoparticles by physical motion in a manner different from the background, making them easier to detect. In a further example, the interrogating signal is an electromagnetic radiation signal. In particular, the interrogating signal may be electromagnetic radiation having a wavelength between about 400 nanometers and about 1600 nanometers. The interrogating signal may, more particularly, comprise electromagnetic radiation having a wavelength between about 500 nanometers and about 1000 nanometers. In examples where the nanoparticle conjugates include a fluorophore, the interrogating signal may therefore be an electromagnetic radiation signal with a wavelength that can excite the fluorophore and penetrate the skin or other tissue and subsurface vasculature (e.g., a wavelength in the range of about 500 to about 1000 nanometers), and the response signal is fluorescence radiation from the fluorophore that can penetrate the subsurface vasculature and tissue to reach the detector.

The program instructions 1270 stored on the computer readable medium 1260 may include instructions to perform or facilitate some or all of the device functionality described herein. For instance, in the illustrated embodiment, program instructions 1270 include a controller module 1272, calculation and decision module 1274 and an alert module 1276.

The controller module 1272 can include instructions for operating the data collection system 1210, for example, the detector 1212 and signal source 1214. For example, the controller 1272 may activate signal source 1214 and/or detector 1212 during each of the pre-set measurement periods. In particular, the controller module 1272 can include instructions for controlling the signal source 1214 to transmit an interrogating signal at preset measurement times and controlling the detector 1212 to receive data representative of response signals transmitted from the portion of subsurface vasculature in response to the interrogating signals transmitted at the preset measurement times.

The controller module 1272 can also include instructions for operating a user interface 1220. For example, controller module 1272 may include instructions for displaying data collected by the data collection system 1210 and analyzed by the calculation and decision module 1274, or for displaying one or more alerts generated by the alert module 1275. Further, controller module 1272 may include instructions to execute certain functions based on inputs accepted by the user interface 1220, such as inputs accepted by one or more buttons disposed on the user interface.

Communication platform 1230 may also be operated by instructions within the controller module 1272, such as instructions for sending and/or receiving information via a wireless antenna, which may be disposed on or in the wearable device 1200. The communication interface 1230 can optionally include one or more oscillators, mixers, frequency injectors, etc. to modulate and/or demodulate information on a carrier frequency to be transmitted and/or received by the antenna. In some examples, the wearable device 1200 is configured to indicate an output from the processor by modulating an impedance of the antenna in a manner that is perceivable by a remote server or other remote computing device.

Calculation and decision module 1272 may include instructions for receiving data from the data collection system 1210 in the form of a responsive signal, analyzing the data to determine if the target analyte is present or absent, quantify the measured physiological parameter(s), such as concentration of a target analyte, and analyzing the data to determine if a medical condition is indicated. In particular, the calculation and decision module 1272 may include instructions for determining, for each preset measurement time, a concentration of a clinically-relevant analyte based on the response signal detected by the detector at that measurement time and determining, for each preset measurement time, whether a medical condition is indicated based on at least the corresponding concentration of the clinically-relevant analyte. The preset measurement times may be set to any period and, in one example, are about one hour apart.

The program instructions of the calculation and decision module 1272 may, in some examples, be stored in a computer-readable medium and executed by a processor located external to the wearable device. For example, the wearable device could be configured to collect certain data regarding physiological parameters from the wearer and then transmit the data to a remote server, which may include a mobile device, a personal computer, the cloud, or any other remote system, for further processing.

The computer readable medium 1260 may further contain other data or information, such as medical and health history of the wearer of the device, that may be useful in determining whether a medical condition is indicated. Further, the computer readable medium 1260 may contain data corresponding to certain analyte baselines, above or below which a medical condition is indicated. The baselines may be pre-stored on the computer readable medium 1260, may be transmitted from a remote source, such as a remote server, or may be generated by the calculation and decision module 1274 itself. The calculation and decision module 1274 may include instructions for generating individual baselines for the wearer of the device based on data collected over a certain number of measurement periods. For example, the calculation and decision module 1274 may generate a baseline concentration of a target blood analyte for each of a plurality of measurement periods by averaging the analyte concentration at each of the measurement periods measured over the course of a few days, and store those baseline concentrations in the computer readable medium 1260 for later comparison. Baselines may also be generated by a remote server and transmitted to the wearable device 1200 via communication interface 1230. The calculation and decision module 1274 may also, upon determining that a medical condition is indicated, generate one or more recommendations for the wearer of the device based, at least in part, on consultation of a clinical protocol. Such recommendations may alternatively be generated by the remote server and transmitted to the wearable device.

In some examples, the collected physiological parameter data, baseline profiles, health state information input by device wearers and generated recommendations and clinical protocols may additionally be input to a cloud network and be made available for download by a wearer's physician. Trend and other analyses may also be performed on the collected data, such as physiological parameter data and health state information, in the cloud computing network and be made available for download by physicians or clinicians.

Further, physiological parameter and health state data from individuals or populations of device wearers may be used by physicians or clinicians in monitoring efficacy of a drug or other treatment. For example, high-density, real-time data may be collected from a population of device wearers who are participating in a clinical study to assess the safety and efficacy of a developmental drug or therapy. Such data may also be used on an individual level to assess a particular wearer's response to a drug or therapy. Based on this data, a physician or clinician may be able to tailor a drug treatment to suit an individual's needs.

In response to a determination by the calculation and decision module 1274 that a medical condition is indicated, the alert module 1276 may generate an alert via the user interface 1220. The alert may include a visual component, such as textual or graphical information displayed on a display, an auditory component (e.g., an alarm sound), and/or tactile component (e.g., a vibration). The textual information may include one or more recommendations, such as a recommendation that the wearer of the device contact a medical professional, seek immediate medical attention, or administer a medication.

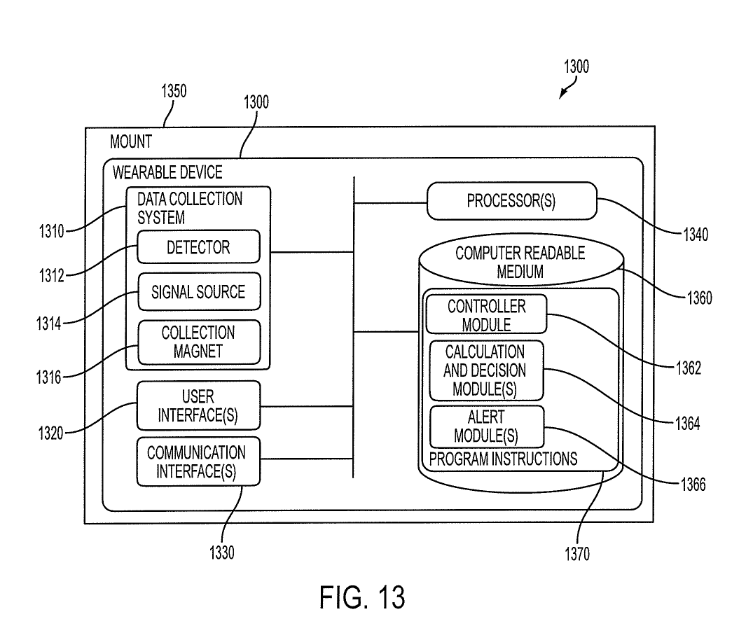

FIG. 13 is a simplified block diagram illustrating the components of a wearable device 1300, according to an example embodiment. Wearable device 1300 is the same as wearable device 1200 in all respects, except that the data collection system 1310 of wearable device 1300 further includes a collection magnet 1316. In this example, the collection magnet 1316 may be used to locally collect magnetic nanoparticles conjugates present in an area of subsurface vasculature proximate to the collection magnet 1316. As described above, collection magnet 1316 is configured to direct a magnetic field into a portion of subsurface vasculature sufficient to cause the magnetic nanoparticles conjugates to collect in a lumen of the portion of subsurface vasculature.

Wearable device 1300 includes a data collection system 1310, which includes a detector 1312, a signal source 1314 (if provided) and a collection magnet 1316, a user interface 1320, a communication interface 1330, a processor 1340 and a computer readable medium 1360 on which program instructions 1370 are stored. All of the components of wearable device 1300 may be provided on a mount 1350. In this example, the program instructions 1370 may include a controller module 1362, a calculation and decision module 1364 and an alert module 1366 which, similar to the example set forth in FIG. 12, include instructions to perform or facilitate some or all of the device functionality described herein. Controller module 1362 further includes instructions for operating collection magnet 1316. For example, controller module 1362 may include instructions for activating collection magnet during a measurement period, for a certain amount of time.

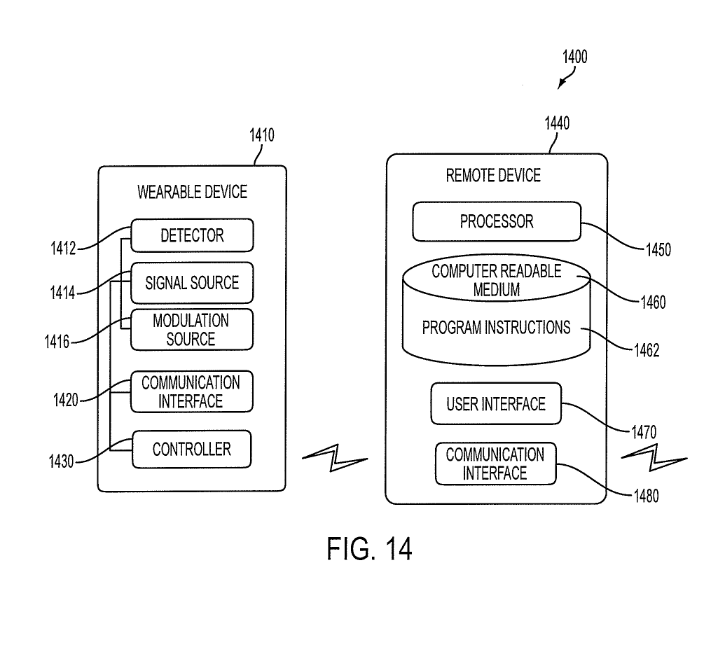

FIG. 14 is a simplified block diagram illustrating the components of an example system 1400, including a wearable device 1410. Wearable device 1410 may take the form of or be similar to one of the wrist-mounted devices 600, 700, 800, 900, or 1000, shown in FIGS. 6A-B, 7A-7C, 8A-8C, 9, and 10. However, wearable device 1410 may also take other forms, such as an ankle, waist, ear, eye or chest-mounted device. Further, any of devices 600, 700, 800, 900, and 1000 may be configured similar to or include any of the components of system 1400, including wearable device 1410.

In particular, FIG. 14 shows an example of a system 1400 including a wearable device 1410 having a detector 1412, in some examples, a signal source 1414, a modulation source 1416, and a communication interface 1420, controlled by a controller 1430. Communication interface 1420 may include an antenna. The components of the wearable device 1410 may be disposed on a mount (not shown) for mounting the device to an external body surface where a portion of subsurface vasculature is readily observable. System 1400 may further include a remote device 1440 in communication with the wearable device 1410, including a processor 1450, a computer readable medium 1460, a user interface 1470, and a communication interface 1480 for communicating with the wearable device 1410 and/or for transmitting data to a server or other remote computing device. While FIG. 14 depicts various components of system 1400 disposed on the wearable device 1410 or the remote device 1440, one of ordinary skill in the art would understand that different configurations and designs are possible, including where all of the components are provided on the wearable device.

Processor 1450 may be a general-purpose processor or a special purpose processor (e.g., digital signal processors, application specific integrated circuits, etc.) and can be configured to execute computer-readable program instructions 1462 that are stored in the computer readable medium 1460 and are executable to provide the functionality of a system 1400 as described herein. The computer readable medium 1460 may include or take the form of one or more non-transitory, computer-readable storage media that can be read or accessed by the processor 1450, and can include volatile and/or non-volatile storage components, such as optical, magnetic, organic or other memory or disc storage, which can be integrated in whole or in part with the processor 1450. The controller 1430 may be configured to operate one or more of the detector 1412, signal source 1414 and modulation source 1416. For example, the controller 1430 may activate the detector 1412, signal source 1414 and modulation source 1416 during each of the pre-set measurement periods.

The program instructions 1462 stored on the computer readable medium 1460 may include instructions to perform or facilitate some or all of the system functionality described herein. For instance, in the illustrated embodiment, program instructions 1462 may include instructions for controller 1430 to operate the detector 1412, signal source 1414 and modulation source 1416. Program instructions 1462 may further cause the processor 1450 to detect the one or more target analytes by differentiating the analyte response signal from the background signal based, at least in part, on a modulation applied by the modulation source 1416. In some cases, the processor may further be configured to differentiate the analyte response signal from the unbound particle signal. Further, the processor 1450 may be configured to determine the concentration of a particular target analyte in the blood from, at least in part, the analyte response signal. The detection and concentration data processed by the processor may be communicated to the patient, for example via the user interface 1470, transmitted to medical or clinical personnel, locally stored or transmitted to a remote server, the cloud, and/or any other system where the data may be stored or accessed at a later time. The program instructions 1462 may also include instructions for operating a user interface 1470, for example, instructions for displaying data transmitted from the wearable device 1410 and analyzed by the processor 1450, or for generating one or more alerts.

### V. Illustrative Methods for Operation of a Wearable Device

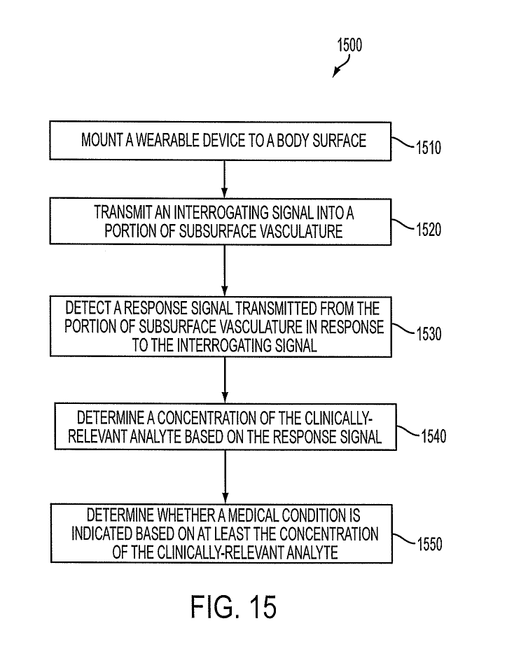

FIG. 15 is a flowchart of a method 1500 for operating a wearable device to take non-invasive, in vivo, real-time measurements of physiological parameters. A wearable device is first mounted to a body surface of a human subject, wherein the body surface is proximate to a portion of subsurface vasculature (1510). In some examples, the wearable device, via a signal source, transmits an interrogating signal into the portion of subsurface vasculature (1520). The wearable device, via a detector, then detects a response signal transmitted from the portion of subsurface vasculature, wherein the response signal is related to binding of a clinically-relevant analyte to nanoparticle conjugates present in a lumen of the subsurface vasculature (1530). In some examples, the response signal is generated in response to an interrogating signal. The nanoparticle conjugates are configured to bind to the clinically-relevant analyte and comprise one or more types of targeting entities such as an antibody or an aptamer. The term “bind” is understood in its broadest sense to also include any detectable interaction between the clinically relevant analyte and the nanoparticle conjugates. The wearable device then determines the presence, absence and/or a concentration of the clinically-relevant analyte based on the response signal (1540) and whether a medical condition is indicated based on at least the presence, absence and/or concentration of the clinically-relevant analyte (1550). Further, in examples where the nanoparticle conjugates are magnetic, the wearable device may further direct a magnetic field into the portion of subsurface vasculature, the magnetic field being sufficient to cause the magnetic nanoparticle conjugates to collect in a lumen of the portion of subsurface vasculature.

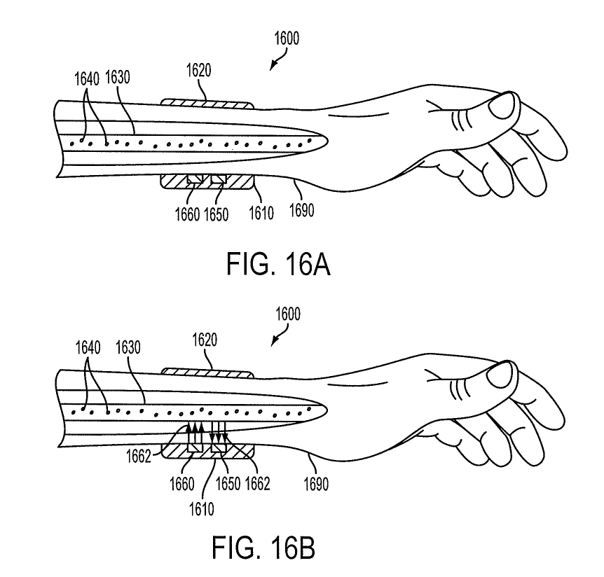

FIGS. 16A-16B, 17A-17B, and 18A-18B are partial cross-sectional side views of a human wrist illustrating the operation of various examples of a wrist-mounted device. In the example shown in FIGS. 16A and 16B, the wrist-mounted device 1600 includes a measurement platform 1610 mounted on a strap or wrist-band 1620 and oriented on the anterior side 1690 of the wearer's wrist. Measurement platform 1610 is positioned over a portion of the wrist where subsurface vasculature 1630 is easily observable. Nanoparticle conjugates 1640 have been introduced into a lumen of the subsurface vasculature by one of the means discussed above. In this example, measurement platform 1610 includes a data collection system having both a detector 1650 and a signal source 1660. FIG. 16A illustrates the state of the subsurface vasculature when measurement device 1600 is inactive. The state of the subsurface vasculature during a measurement period is illustrated in FIG. 16B. At this time, signal source 1660 is transmitting an interrogating signal 1662 into the portion of subsurface vasculature and detector 1650 is receiving a response signal 1652 generated in response to the interrogating signal 1662. The response signal 1652 is related to the binding of a clinically relevant analyte present in the subsurface vasculature to the nanoparticle conjugates 1640. As described above, in some embodiments, an interrogating signal may not be necessary to generate a response signal related to the binding of an analyte to the nanoparticle conjugates.

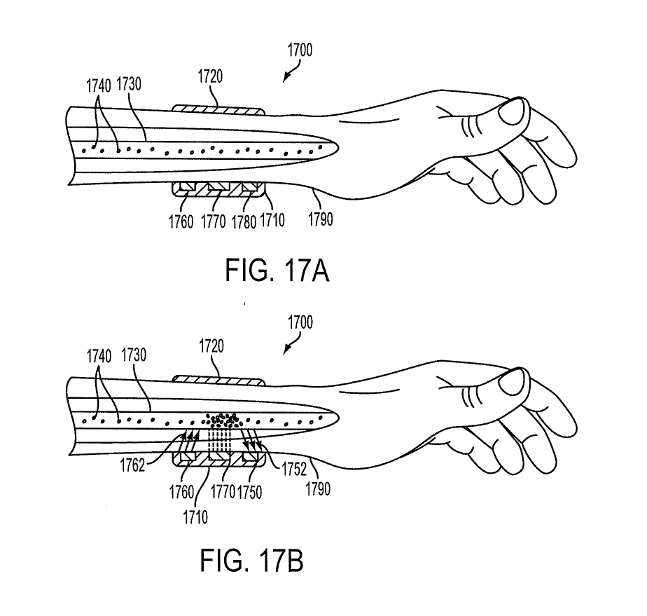

Similar to the system depicted in FIGS. 16A and 16B, FIGS. 17A and 17B illustrate a wrist-mounted device 1700 including a measurement platform 1710 mounted on a strap or wristband 1720 and oriented on the anterior side 1790 of the wearer's wrist. In this example, measurement platform 1710 includes a data collection system having a detector 1750, a signal source 1760 and a collection magnet 1770. FIG. 17A illustrates the state of the subsurface vasculature when measurement device 1700 is inactive. The state of the subsurface vasculature when measurement device 1700 is active during a measurement period is illustrated in FIG. 17B. At this time, collection magnet 1770 generates a magnetic field 1772 sufficient to cause magnetic nanoparticle conjugates 1740 present in a lumen of the subsurface vasculature 1730 to collection in a region proximal to the magnet 1770. Signal source 1760 transmits an interrogating signal 1762 into the portion of subsurface vasculature and detector 1750 is receiving a response signal 1752 generated in response to the interrogating signal 1762. The response signal 1752 is related to the binding of a clinically relevant analyte present in the subsurface vasculature to the magnetic nanoparticle conjugates 1740. As described above, in some embodiments, an interrogating signal may not be necessary to generate a response signal related to the binding of an analyte to the magnetic nanoparticle conjugates.

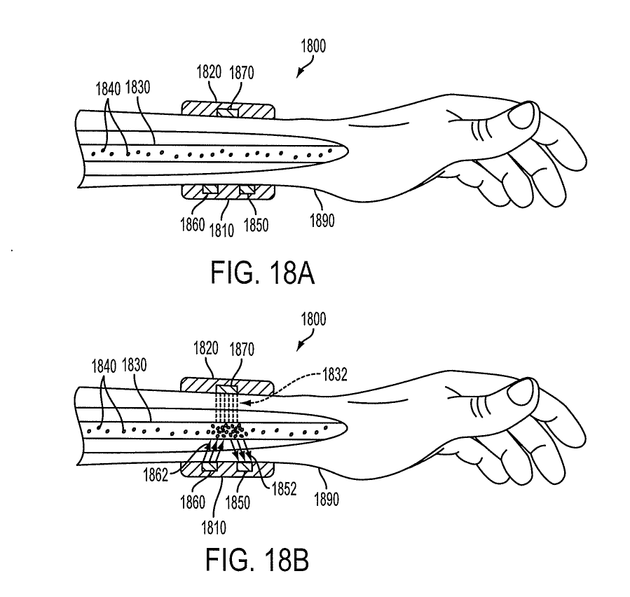

FIGS. 18A and 18B illustrate a further embodiment of a wrist-mounted device 1800 having a measurement platform 1810 disposed on a strap 1820, wherein the detector 1850 and signal source 1860 are positioned on the posterior side 1890 of the wearer's wrist and the collection magnet 1870 is disposed on the anterior side 1880 of the wearer's wrist. Similar to the embodiments discussed above, FIG. 18A illustrates the state of the subsurface vasculature when measurement device 1800 is inactive. The state of the subsurface vasculature when measurement device 1800 is active during a measurement period is illustrated in FIG. 18B. At this time, collection magnet 1870 generates a magnetic field 1832 sufficient to cause magnetic nanoparticle conjugates 1840 present in a lumen of the subsurface vasculature 1830 to collection in a region proximal to the magnet 1870. Signal source 1860 transmits an interrogating signal 1862 into the portion of subsurface vasculature and detector 1850 is receiving a response signal 1852 generated in response to the interrogating signal 1862. The response signal 1852 is related to the binding of a clinically relevant analyte present in the subsurface vasculature to the magnetic nanoparticle conjugates 1840. As described above, in some embodiments, an interrogating signal may not be necessary to generate a response signal related to the binding of an analyte to the magnetic nanoparticle conjugates.

Both FIGS. 17B and 18B illustrate the path of the interrogating signal (1762, 1862) transmitted by the signal source (1760, 1860) and the responsive signal (1752, 1852) detected by the detector (1750, 1850) essentially overlapping over a portion of subsurface vasculature. In some examples, the signal source (1760, 1860) and the detector (1750, 1850) may be angled towards each other so that they are interrogating and detecting from essentially the same area of subsurface vasculature. However, in some instances, such as in the example shown in FIG. 15B, the paths of the interrogating signal (1762, 1862) transmitted by the signal source (1760, 1860) and the responsive signal (1752, 1852) detected by the detector (1750, 1850) may not overlap.

### VI. Illustrative Methods for Real-Time, High-Density Physiological Data Collection Using a Wrist Mounted Device

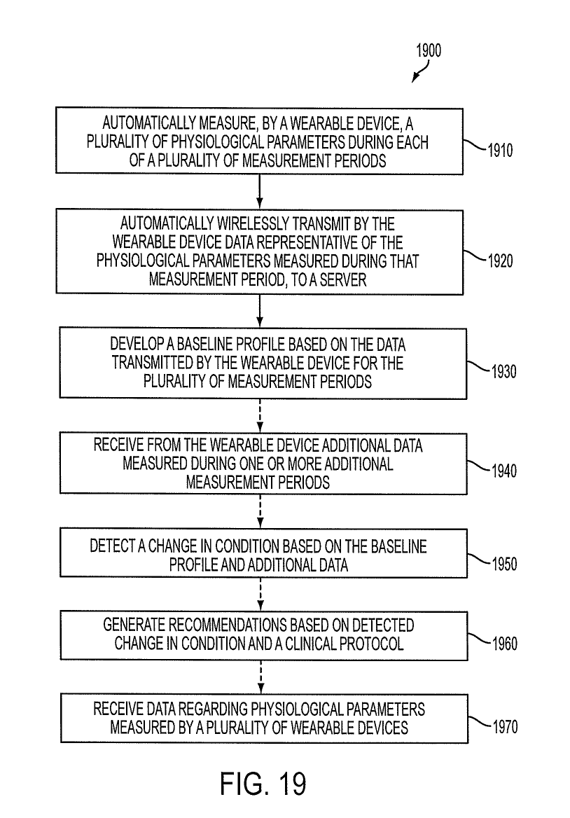

FIG. 19 is a flowchart of a method 1900 for using a wearable device to take real-time, high-density, non-invasive, in vivo measurements of physiological parameters. In a first step, the wearable device automatically measures one or more physiological parameters during each of a plurality of measurement periods (1910). The length of the measurement period may be set on the device itself or may be set remotely, for example, by instruction from a remote server. The device may be configured with many measurement periods each day—for example, continuous, every second, every minute, every hour, every 6 hours, etc.—or may be configured to take measurements once a week or once a month. Further, a different measurement period may be set for each of the physiological parameters being measured. The measurement periods may extend through a plurality of consecutive days and each of the consecutive days may include multiple measurement periods. Each of the consecutive days may further include at least twenty-four measurement periods and the plurality of consecutive days may include at least thirty days. At least some of the physiological parameters are measured by non-invasively detecting one or more analytes in blood circulating in subsurface vasculature proximate to the wearable device.

After conclusion of a measurement period, for each of the plurality of measurement periods, the wearable device transmits to a server data representative of the physiological parameters measured during that measurement period (1920). The wearable device may be configured to automatically transmit the data to a server, may be configured to transmit on command of the wearer, or may be configured to transmit on instruction from a remote server. Further, the device may be configured to automatically transmit the data at the end of each measurement period, or at some more frequent or infrequent rate. For example, the device could be configured to transmit every five minutes, at the end of each day, at the end of the month, at nighttime only, etc.

In response, the server is configured to develop a baseline profile based on the data transmitted by the wearable device for the plurality of measurement periods (1930). In some embodiments, the baseline profile includes an individual baseline profile based on the data transmitted by the wearable device for the plurality of measurement periods for an individual user wearing the wearable device. As described above, the baseline profile may include patterns for how one or more of the wearer's physiological parameters typically change over time, such as during the course of a day, a week, or a month. The baseline profile may further include threshold values of certain target analytes, above or below which a medical condition may be indicated.

After the server has developed an individual baseline profile for a wearer of the device, the server may receive additional data regarding the physiological parameters from the wearable device measured during one or more additional measurement periods (1940). The server may then compare the additional data, collected over additional measurement periods, to the individual baseline profile. If the additional data is consistent with the patterns embodied in the individual baseline profile, the server may determine that the wearer's condition has not changed. On the other hand, if the additional data deviates from the patterns embodied in the baseline profile, the server may detect a change in the wearer's condition (1950). The change in condition could, for example, indicate that the wearer has developed a disease, disorder, or other adverse medical condition or may be at risk for a severe medical condition, such as a stroke or a heart attack, in the near future.

If the server detects a change in condition based on the individual baseline profile and the additional data, it may generate one or more recommendations based on the detected change in condition and a clinical protocol (1960). For example, the server may generate a recommendation that the wearer take a particular medication or supplement, schedule an appointment with a medical professional, go to the hospital to seek immediate medical attention, abstain from certain activities, etc. The server may also be configured to receive data regarding physiological parameters measured by a plurality of wearable devices (1970) and use that data to develop, at least in part, the clinical protocol. The clinical protocol may also be developed based, at least in part, on any known health information or medical history of the wearer, and/or on recognized standards of care in the medical field. The wearable device may receive the one or more recommendations generated by the server (1970) and provide an indication of the one or more recommendations via a user interface on the wearable device.

In some embodiments, the server may be configured to receive data regarding physiological parameters measured by a plurality of wearable devices. The server may use this data collected from a plurality of wearable devices—worn by a plurality of users—to develop, at least in part, a population baseline profile. Such population baseline profiles may be used, for example, for comparison with an individual's baseline profile. Those of skill in the art will readily recognize that comparison of an individual's physiological parameters measured over time to that individual's own baseline may not be sufficient to recognize an abnormality in that physiological parameter. For example, while a physiological parameter for an individual wearer of the device may not deviate from that individual's baseline, that individual baseline may be well above the population baseline generated from data collected from a plurality of wearers of the device. Thus, comparison to what is “normal” or “average” for a population may be necessary for effective identification or prevention of a medical condition in an individual.

Accordingly, the server may further be configured to receive from the wearable device additional data measured during one or more additional measurement periods, detect a change in condition based on the population baseline profile and the additional data, and generate one or more recommendations based on the detected change in condition and a clinical protocol. The wearable device may receive the one or more recommendations generated by the server and provide an indication of the one or more recommendations via a user interface on the wearable device.

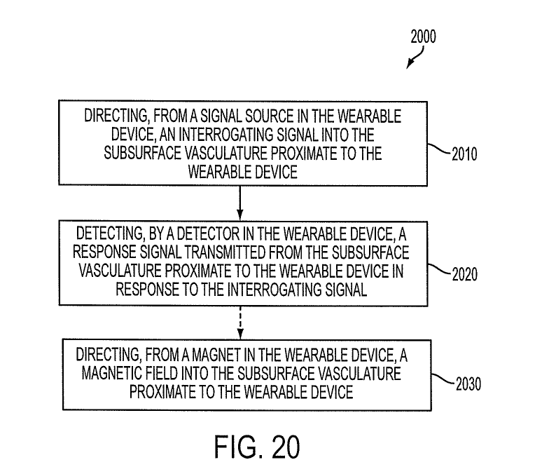

In further embodiments, the method may include introducing nanoparticle conjugates into the blood, wherein the nanoparticle conjugates are configured to bind to the one or more analytes. As shown in FIG. 20, the wearable device may non-invasively measure one or more analytes in blood circulating in subsurface vasculature proximate to the wearable device by directing, from a signal source in the wearable device, an interrogating signal into the subsurface vasculature proximate to the wearable device (2010). As discussed above, this step may not be necessary in cases where the nanoparticle conjugates generate a response signal related to binding of the one or more analytes without the need for an interrogating signal. In any case, the wearable device may detect, with a detector, a response signal transmitted from the subsurface vasculature proximate to the wearable device in response to the interrogating signal (2020). The response signal is related to binding of the one or more analytes to the nanoparticle conjugates. In examples where an interrogating signal is used, the interrogating signal may include a time-varying magnetic field and the response signal may include an externally-detectable physical motion due to the time-varying magnetic field. The interrogating signal may include an electromagnetic pulse (e.g., a radio frequency (RF) pulse) and the response signal may include a magnetic resonance (MR) signal. The interrogating signal may include electromagnetic radiation having a wavelength between about 400 nanometers and about 1600 nanometers, more particularly, a wavelength between about 500 nanometers and about 1000 nanometers. Where the nanoparticle conjugates also include a fluorophore, the response signal may include fluorescence radiation transmitted by the fluorophore in response to the interrogating signal.

In some examples, the nanoparticle conjugates may also be magnetic. The process of measuring one or more analytes in blood circulating in subsurface vasculature may further include directing, from a magnet in the wearable device, a magnetic field into the subsurface vasculature proximate to the wearable device (2030). The magnetic field is sufficient to cause the magnetic nanoparticle conjugates to collect in a lumen of the subsurface vasculature proximate to the wearable device.

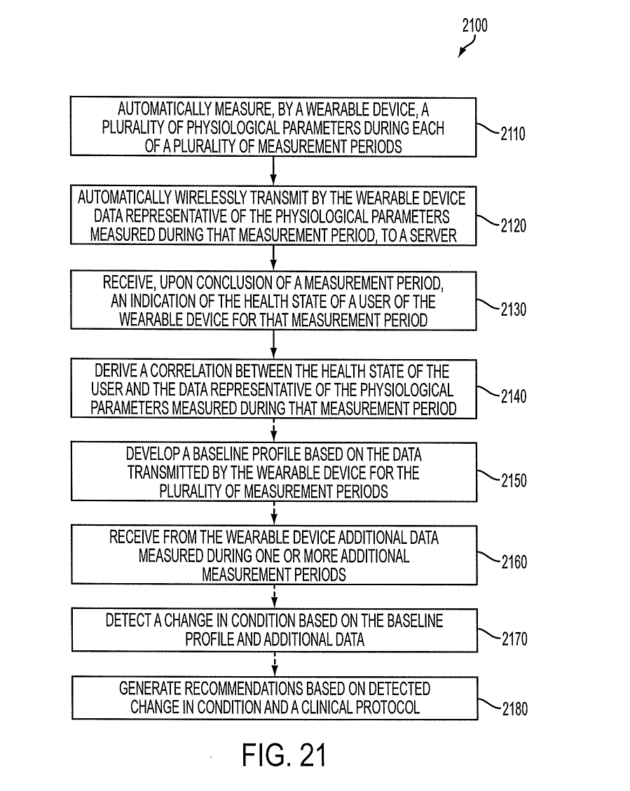

FIG. 21 is a flowchart of a method 2100 for using a wearable device to take real-time, high-density, non-invasive, in vivo measurements of physiological parameters. In a first step, the wearable device automatically measures one or more physiological parameters during each of a plurality of measurement periods (2110). The measurement periods may extend through a plurality of consecutive days, wherein each of the consecutive days includes multiple measurement periods. At least some of the physiological parameters are measured by non-invasively detecting one or more analytes in blood circulating in subsurface vasculature proximate to the wearable device.

Upon conclusion of a measurement period for each of the plurality of measurement periods, the wearable device automatically wirelessly transmits to a server data representative of the physiological parameters measured during that measurement period (2120). The server may be configured to receive, upon conclusion of a measurement period, an indication of the health state of a user of the wearable device for that measurement period (2130) and derive a correlation between the health state of the user and the data representative of the physiological parameters measured during that measurement period (2140). For example, the server may be configured to recognize patterns, for example, every time a physiological parameter reaches or drops to a certain level, the wearer of the device indicates that he or she experiences a migraine. Recognition of these patterns or correlations may help medical professionals to recognize, prevent, diagnose and/or treat of health conditions in that individual. Further, the server may be configured to use these correlations to alert the user that a medical condition may be imminent.

A baseline profile may be developed by the server based on the data transmitted by the wearable device for the plurality of measurement periods (2150). The server may further be configured to receive from the wearable device additional data representative of the physiological parameters measured during one or more additional measurement periods (2160), detect a change in condition based on the baseline profile and the additional data (2170), and generate one or more recommendations based on the detected change in condition and a clinical protocol (2180). The clinical protocol may be developed based, at least in part, on the derived correlation. For example, the clinical protocol may indicate that a medical condition may be imminent based on a comparison between current measurement of a physiological parameter and the derived correlation between previously measured physiological parameters and previously reported health state.

In a further example, the server may be configured to receive data regarding physiological parameters measured by a plurality of wearable devices and receive an indication of the health state of the users of the plurality of wearable devices for a plurality of measurement periods. The server may then derive a correlation between the health state of the users and the data representative of the physiological parameters measured during the plurality of measurement periods. Population data of this kind may be significant in that such correlations may never before have been drawn between that physiological parameter and a particular health condition. Such correlations may be used in prediction, prevention, diagnoses and treatment of health conditions. The server may also be configured to receive from the wearable device additional data representative of the physiological parameters measured during one or more additional measurement periods and generate one or more recommendations based on the received additional data and a clinical protocol, wherein the clinical protocol is developed based, at least in part, on the derived correlation.

In a further example, the wearable device itself may be configured to perform the steps described above as being performed by a remote server. For example, the wearable device may be configured to analyze the data representative of the physiological parameters, generate a baseline profile, compare data collected from additional measurement periods to the baseline profile, and generate recommendations based on a clinical protocol. The wearable device may further be configured to transmit, either automatically or on some other frequency, certain data to the remote server.

### VII. Conclusion

Where example embodiments involve information related to a person or a device of a person, some embodiments may include privacy controls. Such privacy controls may include, at least, anonymization of device identifiers, transparency and user controls, including functionality that would enable users to modify or delete information relating to the user's use of a product.

Further, in situations in where embodiments discussed herein collect personal information about users, or may make use of personal information, the users may be provided with an opportunity to control whether programs or features collect user information (e.g., information about a user's medical history, social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1. A composition comprising:
a nanoparticle;
first oligonucleotides of one or more types that are bound to the nanoparticle, each type of first oligonucleotide having a sequence; and
targeting conjugates of one or more types, each type of targeting conjugate comprising a targeting entity and a second oligonucleotide bound to the targeting entity and having a sequence that is complementary to a sequence of a predetermined type of the first oligonucleotides, wherein the second oligonucleotide is covalently bound to a surface of the nanoparticle, a functional group on the surface of the nanoparticle, or one of the first oligonucleotides.

2. The composition of claim 1, wherein the composition includes two or more types of targeting conjugates and two or more types of first oligonucleotides.

3. The composition of claim 1, wherein the composition includes a single type of first oligonucleotide and two or more types of targeting conjugates.

4. The composition of claim 1, wherein the targeting entity comprises a member of a specific binding pair.

5. A library comprising nanoparticle conjugates of one or more types, each type of nanoparticle conjugate comprising:
a nanoparticle;
first oligonucleotides of one or more types that are bound to the nanoparticle, each type of first oligonucleotide having a sequence; and
targeting conjugates of one or more types, each type of targeting conjugate comprising a targeting entity and a second oligonucleotide bound to the targeting entity and having a sequence that is complementary to a sequence of a predetermined type of the first oligonucleotides, wherein the second oligonucleotide is covalently bound to a surface of the nanoparticle, a functional group on the surface of the nanoparticle, or one of the first oligonucleotides.

6. A method comprising providing a first nanoparticle conjugate comprising a nanoparticle and first oligonucleotides of one or more types bound to the nanoparticle, each type of first oligonucleotide having a sequence;
(a) contacting the first nanoparticle conjugate with targeting conjugates of one or more types, each type of targeting conjugate comprising a targeting entity and a second oligonucleotide bound to the targeting entity, wherein the contacting occurs under suitable hybridization conditions to form a second nanoparticle conjugate, wherein the second oligonucleotide has a sequence that is complementary to a sequence of the first oligonucleotides and wherein the second oligonucleotide includes a reactive group that binds to a surface of the nanoparticle, a functional group on the nanoparticle, or one of the first oligonucleotides;
(b) washing the second nanoparticle conjugate; and
(c) covalently reacting the reactive group of the second oligonucleotide of the second nanoparticle conjugate to the surface of the nanoparticle, a functional group on the nanoparticle, or one of the first oligonucleotides under suitable reaction conditions to form a third nanoparticle conjugate.

7. The composition of claim 6, wherein the second oligonucleotides are covalently bound to the surface or a functional group on the surface of the third nanoparticle conjugate.

8. A method comprising:
(a) providing a first nanoparticle conjugate comprising a nanoparticle and a first oligonucleotide bound to the nanoparticle;
(b) contacting the first nanoparticle conjugate with second oligonucleotides of one or more types, each type of second oligonucleotide comprising a targeting sequence and a sequence that is complementary to a sequence of the first oligonucleotide to produce a second nanoparticle conjugate, wherein said contacting occurs under suitable hybridization conditions;
(c) extending the first oligonucleotide under suitable polymerase chain reaction conditions to produce an extended first oligonucleotide comprising a complementary targeting sequence that is complementary to the targeting sequence of the second oligonucleotide;
(d) removing the second oligonucleotide from the second nanoparticle under suitable dehybridization conditions to produce a third nanoparticle conjugate;
(e) washing the third nanoparticle conjugate;
(f) contacting the third nanoparticle conjugate with a targeting conjugate of one or more types, each type of targeting conjugate comprising a targeting entity and a third oligonucleotide that is bound to the targeting entity, wherein the third oligonucleotide has a predetermined sequence that is complementary to the complementary targeting sequence of the extended first oligonucleotide, wherein the third oligonucleotide has a reactive group that is capable of covalently binding to a surface of the nanoparticle, a functional group on the nanoparticle, or the first oligonucleotide and wherein said contacting occurs under hybridization conditions to form a fourth nanoparticle conjugate.

9. The method of claim 8, further comprising;
(f1) covalently binding the third oligonucleotide to the surface of the nanoparticle, to a functional group on the nanoparticle, or to the first oligonucleotide to form a nanoparticle conjugate probe.

10. The method of claim 8, further comprising;
(g) extending the third oligonucleotide under suitable polymerase chain reaction conditions to produce a modified third oligonucleotide comprising a sequence that is complementary to the sequence of the first oligonucleotide; and
(h) covalently reacting the reactive group of the modified third oligonucleotide with the surface of the nanoparticle or a functional group on the nanoparticle to form a nanoparticle conjugate probe.

11. The method of claim 8, wherein the method involves a single type of second oligonucleotide and a single type of targeting conjugate.

12. The method of claim 8, wherein the method involves two or more types of second oligonucleotide and two or more types of targeting conjugates.

13. A method comprising:
(a) providing a first nanoparticle conjugate comprising a nanoparticle and a first oligonucleotide bound to the nanoparticle;
(b) contacting the first nanoparticle conjugate with a second oligonucleotide of two or more types, each type of second oligonucleotide comprising a targeting sequence and a sequence that is complementary to a sequence of the first oligonucleotide to produce a second nanoparticle conjugate, wherein said contacting occurs under suitable hybridization conditions;
(c) washing the second nanoparticle conjugate;
(d) contacting the second nanoparticle conjugate with targeting conjugates of one or more types, each type of targeting conjugate comprising a targeting entity and a third oligonucleotide that is bound to the targeting entity, the third oligonucleotide having a predetermined sequence that is complementary to the targeting sequence of the second oligonucleotide, wherein said contacting occurs under suitable hybridization conditions to form a third nanoparticle conjugate; and
(e) ligating the third oligonucleotide to the first oligonucleotide under suitable ligation conditions to form a fourth nanoparticle conjugate.

14. The method of claim 13, further comprising removing the second oligonucleotide of the fourth nanoparticle conjugate under suitable dehybridization conditions to form a nanoparticle conjugate probe.

15. The method of claim 13, further comprising covalently binding the second oligonucleotide of the fourth nanoparticle conjugate to form a nanoparticle conjugate probe.

16. The method of claim 13, wherein the method involves a single type of second oligonucleotide and a single type of targeting conjugate.

17. The method of claim 13, wherein the method involves two or more types of second oligonucleotides and two or more types of targeting conjugates.

18. A system comprising a wearable device comprising:
a mount configured to mount the wearable device on an external surface of a living body;
a detector configured to detect an analyte response signal transmitted from tissue through the external surface, wherein the tissue contains a nanoparticle conjugate comprising a nanoparticle, first oligonucleotides of one or more types that are bound to the nanoparticle, each type of first oligonucleotide having a sequence, and targeting conjugates of one or more types, each type of targeting conjugate comprising a targeting entity and a second oligonucleotide bound to the targeting entity and having a sequence that is complementary to the sequence of a predetermined type of the first oligonucleotides, wherein the second oligonucleotide is covalently bound to a surface of the nanoparticle, a functional group on the surface of the nanoparticle, or one of the first oligonucleotides; and
a processor configured to determine a presence or absence of the one or more target analytes based on the analyte response signal.

19. The system of claim 18, further comprising a modulation source configured to modulate the analyte response signal differently than a background signal.

20. The system of claim 19, wherein the processor is configured to differentiate the analyte response signal from the background signal based, at least in part, on the modulation by the modulation source.

21. The system of claim 19, further comprising an interrogating signal source configured to apply an interrogating signal to the tissue, wherein the analyte response signal is transmitted in response to the interrogating signal.

22. The system of claim 21, wherein the modulation source is configured to modulate the analyte response signal by modulating the interrogating signal.

23. The system of claim 18, wherein the tissue comprises subsurface vasculature, and wherein the particles are in blood circulating in the subsurface vasculature.

24. A method comprising:
introducing nanoparticle conjugate probes into the living body, the nanoparticle conjugates probes comprising a nanoparticle, first oligonucleotides of one or more types that are bound to the nanoparticle, each type of first oligonucleotide having a sequence, and targeting conjugates of one or more types, each type of targeting conjugate comprising a targeting entity and a second oligonucleotide bound to the targeting entity and having a sequence that is complementary to a sequence of a predetermined type of the first oligonucleotides, wherein the second oligonucleotide is covalently bound to a surface of the nanoparticle, a functional group on the surface of the nanoparticle, or one of the first oligonucleotides, wherein the nanoparticle conjugate probes are configured to bind with one or more target analytes, wherein presence or absence of the one or more target analytes in the living body is correlated with the biological state of the living body;
detecting, by a wearable device mounted on an external surface of the living body, a signal transmitted from the living body, wherein the signal includes an analyte response signal that is related to binding of the one or more target analytes with the nanoparticle conjugates; and
determining a presence or absence of the one or more target analytes based on the analyte response signal.
